(19) United States
(12) Patent Application Publication
(10) Pub. No.: US 2025/0263658 A1
(43) Pub. Date: Aug. 21, 2025

(54) CELL POPULATIONS ADAPTED TO A TUMOR MICROENVIRONMENT

(71) Applicant: Xcell Biosciences, Inc., San Francisco, CA (US)

(72) Inventors: Yunmin Li, Millbrae, CA (US); Albert Wong, South San Francisco, CA (US); Quynh Anh Thuc Lu, Mountain View, CA (US); James Lim, San Francisco, CA (US)

(21) Appl. No.: 18/904,794

(22) Filed: Oct. 2, 2024

Related U.S. Application Data
(63) Continuation of application No. PCT/US23/17249, filed on Apr. 3, 2023.
(60) Provisional application No. 63/327,780, filed on Apr. 5, 2022.

(51) Int. Cl. C12N 5/0783; A61K 35/17; A61K 40/11; A61K 40/31; C12N 5/00

(52) U.S. Cl. CPC C12N 5/0636; A61K 35/17; A61K 40/11; A61K 40/31; C12N 5/52; C12N 2500/02; C12N 2510/00

(57) ABSTRACT

Provided herein are methods of preparing cell therapies under tumor microenvironment (TME) conditions, for example, hypoxic and hyperbaric conditions, for increased cytotoxicity towards cancer cells. Also disclosed herein are improved cell therapies with increased cytotoxicity towards cancer cells. The methods may include producing a CAR-T cell therapy for increased cytotoxicity towards cancer cells, culturing the population of CAR-T cells in the hypoxic environment and the hyperbaric environment for at least 10 generations to produce the CAR-T cell therapy, and the CAR-T cell therapy comprises increased cytotoxicity towards cancer cells as compared to the population of CAR-T cells.

### CROSS-REFERENCE

This application claims priority to International Application PCT/US23/17249, filed Apr. 3, 2023, which application claims priority to U.S. Provisional Patent Application No. 63/327,780 filed on Apr. 5, 2022, which application is incorporated herein by reference in its entirety for all purposes.

### TECHNICAL FIELD

This application is directed to the development of immunotherapeutic cell populations for treating solid tumors, said populations having potential of enhanced therapeutic effectiveness.

### BACKGROUND

Treatment of solid tumors can be complicated by the tumor microenvironment (TME) which tumors, themselves, create. The tumor microenvironment typically includes intratumor pressure that is higher than the pressure in neighboring body compartments, and a vascular system that is poor, compared to the vascularity of other anatomical sites, thus the tumor microenvironment is typically hyperbaric and hypoxic. The poor vascularity tends to affect the internal portion of tumors adversely, promoting necrosis, which has further morbid consequences for a patient. The higher pressure and poor vascularity of tumors also tends to blunt the effectiveness of blood-borne cancer treatments, whether the treatments include bioactive agents or therapeutic immune cell populations. Innovations that could improve the accessibility and effectiveness of blood-borne anticancer treatments are desirable in formulary of anticancer treatments.

### SUMMARY OF THE INVENTION

It is appreciated by the inventors that cell therapies which are used as treatments for cancer may function less effectively in vivo than in in vitro models due to the inadequacy of in vitro models or in vitro culture environments under which cell therapies are produced to replicate the in vivo tumor microenvironment under which the cell therapies function. Cell therapies which are administered in vivo may thus function less effectively than expected based on in vitro models, or may require an extended adaptation period once they are administered in vivo as to adapt to the tumor microenvironment under which they are expected to function, often to the detriment of the patient who will not obtain the full benefit of the cell therapy during the adaptation period. Accordingly, disclosed herein are methods of preparing cell therapies under tumor microenvironment (TME) conditions, for example, hypoxic and hyperbaric conditions, for increased cytotoxicity towards cancer cells. Also disclosed herein are improved cell therapies with increased cytotoxicity towards cancer cells.

Aspects disclosed herein provide a method of producing a CAR-T cell therapy for increased cytotoxicity towards cancer cells, the method comprising: culturing a population of CAR-T cells in a hypoxic environment and a hyperbaric environment; culturing the population of CAR-T cells in the hypoxic environment and the hyperbaric environment for at least 10 generations to produce the CAR-T cell therapy; wherein the CAR-T cell therapy comprises increased cytotoxicity towards cancer cells as compared to the population of CAR-T cells. In some embodiments, the CAR-T cell therapy is an allogenic CAR-T cell therapy. In some embodiments, the increased cytotoxicity towards cancer cells as compared to the population of CAR-T cells comprises an improvement of at least 15% in cytotoxicity towards cancer cells. In some embodiments, the increased cytotoxicity towards cancer cells as compared to the population of CAR-T cells comprises an improvement of at least 15% in cytotoxicity towards cancer cells. In some embodiments, the increased cytotoxicity towards cancer cells as compared to the population of CAR-T cells comprises an improvement of at least 5% in cytotoxicity towards cancer cells. In some embodiments, the increased cytotoxicity towards cancer cells as compared to the population of CAR-T cells comprises an improvement of at least 10% in cytotoxicity towards cancer cells. In some embodiments, the CAR-T cell therapy further comprises: a) an increased growth rate, (b) an increased viability, or c) both, as compared to the population of CAR-T cells. In some embodiments, the population of CAR-T cells has not previously been cultured in a hypoxic environment or hyperbaric environment. In some embodiments, the method further includes contacting the population of CAR-T cells with a cancer cell during the culturing of a. or b. In some embodiments, the method further includes contacting the population of CAR-T cells with a population of cancer cells during the culturing of a. or b. at a ratio of at least 1:1 (the population of CAR-T cells: the population of cancer cells, respectively). In some embodiments, the method further includes contacting the population of CAR-T cells with a population of cancer cells during the culturing of a. or b. at a ratio of at least 2:1 (the population of CAR-T cells: the population of cancer cells, respectively). In some embodiments, the method further includes contacting the population of CAR-T cells with a population of cancer cells during the culturing of a. or b. at a ratio of at least 5:1 (the population of CAR-T cells: the population of cancer cells, respectively). In some embodiments, the method further includes contacting the population of CAR-T cells with a population of cancer cells during the culturing of a. or b. at a ratio of at 10:1 (the population of CAR-T cells: the population of cancer cells, respectively). In some embodiments, the method further includes contacting the population of CAR-T cells with the population of cancer cells repeatedly. In some embodiments, the method further includes contacting the population of CAR-T cells with the population of cancer cells repeatedly, once at least 25% of the population of the cancer cells has been destroyed by the population of CAR-T cells. In some embodiments, the method further includes contacting the population of CAR-T cells with the population of cancer cells during the culturing of a. or b. at a ratio of 10:1 (the population of CAR-T cells: the population of cancer cells, respectively), at least 4 times during the culturing of a. and b. In some embodiments, the method further includes contacting the population of CAR-T cells with a population of cancer cells during the culturing of a. or b. at a ratio of 10:1 (the population of CAR-T cells: the population of cancer cells, respectively), at intervals of at least 24 hours. In some embodiments, the method further includes contacting the population of CAR-T cells with the population of cancer cells repeatedly. In some embodiments, the method further includes contacting the population of CAR-T cells with the population of cancer cells repeatedly, once at least 35% of the population of the cancer cells has been destroyed by the population of CAR-T cells. In some embodiments, the method further includes contacting the population of CAR-T cells with the population of cancer cells repeatedly, once at least 50% of the population of the cancer cells has been destroyed by the population of CAR-T cells. In some embodiments, the method further includes contacting the population of CAR-T cells with the population of cancer cells repeatedly, once at least 75% of the population of the cancer cells has been destroyed by the population of CAR-T cells. In some embodiments, the method further includes contacting the population of CAR-T cells with the population of cancer cells repeatedly, once at least 98% of the population of the cancer cells has been destroyed by the population of CAR-T cells. In some embodiments, the method further includes contacting the population of CAR-T cells with the population of cancer cells repeatedly, once at least 99% of the population of the cancer cells has been destroyed by the population of CAR-T cells. In some embodiments, the method further includes contacting the population of CAR-T cells with the population of cancer cells repeatedly, once at least 25% of the population of the cancer cells has been destroyed by the population of CAR-T cells. In some embodiments, the method further includes contacting the population of CAR-T cells with a population of cancer cells a second time at about 48 hours. In some embodiments, the method further includes contacting the population of CAR-T cells with a population of cancer cells a third time at about 120 hours. In some embodiments, the method further includes contacting the population of CAR-T cells with a population of cancer cells a fourth time at about 172 hours. In some embodiments, the population of cancer cells correspond to a target cancer cell intended to be treated by the CAR-T cell therapy. In some embodiments, the contacting the population of CAR-T cells with the population of cancer cells increases the cytotoxicity of the cell therapy toward the cancer cell intended to be treated by the CAR-T cell therapy. In some embodiments, the contacting the population of CAR-T cells with the population of cancer cells increases the cytotoxicity of the cell therapy toward cancer cells. In some embodiments, the CAR-T cell therapy further comprises: (a) an increased growth rate, (b) an increased viability, (c) an altered metabolic activity profile, or (d) an altered differentiated functionality, (e) or combinations thereof. In some embodiments, the CAR-T cell therapy is an autologous CAR-T cell therapy. In some embodiments, the hypoxic environment comprises up to 3% wt. O2. In some embodiments, the hypoxic environment comprises up to 2% wt. O2. In some embodiments, the hypoxic environment comprises up to 1% wt. O2. In some embodiments, the hyperbaric environment comprises at least 1 psig. In some embodiments, the hyperbaric environment comprises at least 2 psig. In some embodiments, the hyperbaric environment comprises at least 4 psig. In some embodiments, the hyperbaric environment comprises at least 4 psig. In some embodiments, the CAR-T cell therapy comprises an elevated level of a hypoxia response element activation within a hypoxia-induced factor (HIF) gene. In some embodiments, the CAR-T cell therapy comprises an elevated expression of a hypoxia-induced factor. In some embodiments, the CAR-T cell therapy comprises an elevated expression of a hypoxia-induced factor, wherein the hypoxia-induced factor comprises any one or more of HIF-1α, HIF-1b, HIF-2a, HIF-2b, HIF-1a, or HIF-1b. In some embodiments, the method further includes cryogenically preserving a quantity of the CAR-T cell therapy as a cell bank. In some embodiments, the method further includes thawing the CAR-T cell therapy, culturing the CAR-T cell therapy, administering the CAR-T cell therapy to a subject, or combinations thereof. In some embodiments, the method further includes administering the CAR-T cell therapy to a subject. In some embodiments, the CAR-T cell therapy comprises an increased expression of HIF1α, TRAIL, FasL, GNLY, GZMM, or combinations thereof. In some embodiments, the CAR-T cell therapy comprises a decreased expression of HIF2α, GZMB, Perforin, or combinations thereof. In some embodiments, the CAR-T cell therapy comprises an increased expression of HIF1α, TRAIL, FasL, GNLY, GZMM, or combinations thereof in amounts up to 610% HIF1α, 125% TRAIL, 150% FasL, 300% GNLY, and 300% GZMM of native expression levels in the population of CAR-T cells. In some embodiments, the CAR-T cell therapy comprises an increased expression of HIF1α, TRAIL, FasL, GNLY, GZMM, or combinations thereof in amounts of at least 610% HIF1α, 125% TRAIL, 150% FasL, 300% GNLY, and 300% GZMM of native expression levels in the population of CAR-T cells. In some embodiments, the CAR-T cell therapy comprises a decreased expression of HIF2α, GZMB, Perform, or combinations thereof in approximate amounts of up to 90% HIF2α, 25% GZMB, and 35% Perform of a native level of expression of native expression levels in the population of CAR-T cells. In some embodiments, the CAR-T cell therapy comprises a decreased expression of HIF2α, GZMB, Perforin, or combinations thereof in approximate amounts of at least 90% HIF2α, 25% GZMB, and 35% Perforin of a native level of expression of native expression levels in the population of CAR-T cells. In some embodiments, the CAR-T cell therapy comprises an increased expression of HIF1α, TRAIL, GZMM, or combinations thereof in amounts of up to 290% HIF1α, 150% TRAIL, and 175% GZMM of native expression levels in the population of CAR-T cells. In some embodiments, the CAR-T cell therapy comprises an increased expression of HIF1α, TRAIL, GZMM, or combinations thereof in amounts of at least 290% HIF1α, 150% TRAIL, and 175% GZMM of native expression levels in the population of CAR-T cells. In some embodiments, the CAR-T cell therapy comprises a decreased expression of FasL, Perform, GNYL, or combinations thereof in amounts of up to 33% FasL, 50% Perform, and 80% GNYL of native expression levels in the population of CAR-T cells. In some embodiments, the CAR-T cell therapy comprises a decreased expression of FasL, Perform, GNYL, or combinations thereof in amounts of at least 33% FasL, 50% Perforin, and 80% GNYL of native expression levels in the population of CAR-T cells. In some embodiments, the CAR-T cell therapy comprises an increased expression of HIF1α, TRAIL, FasL, GNLY, GZMM, or combinations thereof. In some embodiments, the CAR-T cell therapy comprises a decreased expression of HIF2α, GZMB, FasL, Perforin, or combinations thereof. In some embodiments, the CAR-T cell therapy comprises increased expression of HIF1α, TRAIL, FasL, GNLY, GZMM, or combinations thereof in amounts of up to 925% HIF1α, 150% TRAIL, 135% FasL, 500% GNLY, and 225% GZMM of native expression levels in the population of CAR-T cells. In some embodiments, the CAR-T cell therapy comprises increased expression of HIF1α, TRAIL, FasL, GNLY, GZMM, or combinations thereof in amounts of at least 925% HIF1α, 150% TRAIL, 135% FasL, 500% GNLY, and 225% GZMM of native expression levels in the population of CAR-T cells. In some embodiments, the CAR-T cell therapy comprises a decreased expression of FasL, Perform, GNYL, GZMM, or combinations thereof in amounts up to 80% HIF2α, 30% Perform, 500% GNYL and 220% GZMM of native expression levels in the population of CAR-T cells. In some embodiments, the CAR-T cell therapy comprises a decreased expression of FasL, Perform, GNYL, GZMM, or combinations thereof in amounts of at least 80% HIF2α, 30% Perform, 500% GNYL and 220% GZMM of native expression levels in the population of CAR-T cells. In some embodiments, the CAR-T cell therapy comprises is short term adapted to hypoxic conditions. In some embodiments, the CAR-T cell therapy is long term adapted to hypoxic conditions. In some embodiments, the CAR-T cell therapy induces cytolysis of cancer cells by death receptor mediated cytotoxicity. In some embodiments, the CAR-T cell therapy induces cytolysis of cancer cells by death receptor mediated cytotoxicity, wherein death receptor mediated cytotoxicity is induced by increased expression of FasL or TRAI1. In some embodiments, the CAR-T cell therapy comprises increased cytolytic activity towards cancer cells comprises inducing cytolysis of at least 75% of cancer cells which contact the CAR-T cell therapy. In some embodiments, the CAR-T cell therapy comprises increased cytolytic activity towards cancer cells comprises inducing cytolysis of at least 85% of cancer cells which contact the CAR-T cell therapy. The method of claim 66, wherein the cancer cells are in vivo. The method of claim 67, wherein the cancer cells are in vivo. In some embodiments, the CAR-T cell therapy comprises a ROR1 CAR. In some embodiments, the CAR-T cell therapy comprises a CD19 CAR. In some embodiments, the CAR-T cell therapy comprises a BCMA CAR. In some embodiments, the CAR-T cell therapy comprises a CD5 CAR. In some embodiments, the CAR-T cell therapy comprises a TRIS CAR. In some embodiments, the CAR-T cell therapy comprises a CD7 CAR. In some embodiments, the CAR-T cell therapy comprises a GPC2 CAR. In some embodiments, the CAR-T cell therapy comprises a NKG2D CAR. In some embodiments, the CAR-T cell therapy comprises a CD14 CAR. In some embodiments, the CAR-T cell therapy comprises an αPD1-MSLN-CAR. In some embodiments, the CAR-T cell therapy comprises a CD33 CAR. In some embodiments, the CAR-T cell therapy comprises a Senl-h19 CAR. In some embodiments, the CAR-T cell therapy comprises an EGFR CAR. In some embodiments, the CAR-T cell therapy comprises an EGFR806 CAR. In some embodiments, the CAR-T cell therapy comprises a GPC3CAR. In some embodiments, the culturing of the population of CAR-T cells in the hypoxic environment and the hyperbaric environment occurs for at least 15 generations to produce the CAR-T cell therapy. In some embodiments, the culturing of the population of CAR-T cells in the hypoxic environment and the hyperbaric environment occurs for at least 20 generations to produce the CAR-T cell therapy. In some embodiments, the culturing of the population of CAR-T cells in the hypoxic environment and the hyperbaric environment occurs for at least 25 generations to produce the CAR-T cell therapy. In some embodiments, the culturing of the population of CAR-T cells in the hypoxic environment and the hyperbaric environment occurs for at least 50 generations to produce the CAR-T cell therapy. In some embodiments, the culturing of the population of CAR-T cells in the hypoxic environment and the hyperbaric environment occurs for at least 75 generations to produce the CAR-T cell therapy. In some embodiments, the culturing of the population of CAR-T cells in the hypoxic environment and the hyperbaric environment occurs for at least 100 generations to produce the CAR-T cell therapy. In some embodiments, the culturing of the population of CAR-T cells in the hypoxic environment and the hyperbaric environment occurs for at least 500 generations to produce the CAR-T cell therapy. In some embodiments, the culturing of the population of CAR-T cells in the hypoxic environment and the hyperbaric environment occurs for at least 1000 generations to produce the CAR-T cell therapy.

Aspects disclosed herein provide a CAR-T cell therapy adapted to a hypoxic environment and a hyperbaric environment comprises increased cytotoxicity towards cancer cells as compared to a population of the same CAR-T cells which are not adapted to the hypoxic environment and the hyperbaric environment. In some embodiments, the CAR-T cell therapy is an allogenic CAR-T cell therapy. In some embodiments, the increased cytotoxicity towards cancer cells as compared to the population of CAR-T cells comprises an improvement of at least 15% in cytotoxicity towards cancer cells. In some embodiments, the increased cytotoxicity towards cancer cells as compared to the population of CAR-T cells comprises an improvement of at least 5% in cytotoxicity towards cancer cells. In some embodiments, the increased cytotoxicity towards cancer cells as compared to the population of CAR-T cells comprises an improvement of at least 10% in cytotoxicity towards cancer cells. In some embodiments, the CAR-T cell therapy further comprises: a) an increased growth rate, (b) an increased viability, or c) both, as compared to the population of CAR-T cells. In some embodiments, the CAR-T cell therapy has previously been contacted with a cancer cell. In some embodiments, the CAR-T cell therapy has previously been contacted with a cancer cell. In some embodiments, the CAR-T cell therapy is short term adapted to hypoxic conditions In some embodiments, the CAR-T cell therapy is long term adapted to hypoxic conditions. In some embodiments, the hypoxic environment comprises up to 3% wt. O2. In some embodiments, the hypoxic environment comprises up to 2% wt. O2. In some embodiments, the hypoxic environment comprises up to 1% wt. O2. In some embodiments, the hyperbaric environment comprises at least 1 psig. In some embodiments, the hyperbaric environment comprises at least 2 psig. In some embodiments, the hyperbaric environment comprises at least 4 psig. In some embodiments, the hyperbaric environment comprises at least 4 psig. In some embodiments, the CAR-T cell therapy comprises an elevated level of a hypoxia response element activation within a hypoxia-induced factor (HIF) gene. In some embodiments, the CAR-T cell therapy comprises an elevated expression of a hypoxia-induced factor. In some embodiments, the CAR-T cell therapy comprises an elevated expression of a hypoxia-induced factor, wherein the hypoxia-induced factor comprises any one or more of HIF-1a, HIF-1b, HIF-2a, HIF-2b, HIF-1a, or HIF-1b. In some embodiments, the CAR-T cell therapy comprises an increased expression of HIF1α, TRAIL, FasL, GNLY, GZMM, or combinations thereof. In some embodiments, the CAR-T cell therapy comprises a decreased expression of HIF2α, GZMB, Perform, or combinations thereof. In some embodiments, the CAR-T cell therapy comprises an increased expression of HIF1α, TRAIL, FasL, GNLY, GZMM, or combinations thereof in amounts up to 610% HIF1α, 125% TRAIL, 150% FasL, 300% GNLY, and 300% GZMM of native expression levels in the population of CAR-T cells which are not adapted to the hypoxic environment and the hyperbaric environment. In some embodiments, the CAR-T cell therapy comprises an increased expression of HIF1α, TRAIL, FasL, GNLY, GZMM, or combinations thereof in amounts of at least 610% HIF1α, 125% TRAIL, 150% FasL, 300% GNLY, and 300% GZMM of native expression levels in the population of CAR-T cells which are not adapted to the hypoxic environment and the hyperbaric environment. In some embodiments, the CAR-T cell therapy comprises a decreased expression of HIF2α, GZMB, Perforin, or combinations thereof in approximate amounts of up to 90% HIF2α, 25% GZMB, and 35% Perform of a native level of expression of native expression levels in the population of CAR-T cells which are not adapted to the hypoxic environment and the hyperbaric environment. In some embodiments, the CAR-T cell therapy comprises a decreased expression of HIF2α, GZMB, Perform, or combinations thereof in approximate amounts of at least 90% HIF2α, 25% GZMB, and 35% Perform of a native level of expression of native expression levels in the population of CAR-T cells which are not adapted to the hypoxic environment and the hyperbaric environment. In some embodiments, the CAR-T cell therapy comprises an increased expression of HIF1α, TRAIL, GZMM, or combinations thereof in amounts of up to 290% HIF1α, 150% TRAIL, and 175% GZMM of native expression levels in the population of CAR-T cells which are not adapted to the hypoxic environment and the hyperbaric environment. In some embodiments, the CAR-T cell therapy comprises an increased expression of HIF1α, TRAIL, GZMM, or combinations thereof in amounts of at least 290% HIF1α, 150% TRAIL, and 175% GZMM of native expression levels in the population of CAR-T cells which are not adapted to the hypoxic environment and the hyperbaric environment. In some embodiments, the CAR-T cell therapy comprises a decreased expression of FasL, Perform, GNYL, or combinations thereof in amounts of up to 33% FasL, 50% Perform, and 80% GNYL of native expression levels in the population of CAR-T cells which are not adapted to the hypoxic environment and the hyperbaric environment. In some embodiments, the CAR-T cell therapy comprises a decreased expression of FasL, Perform, GNYL, or combinations thereof in amounts of at least 33% FasL, 50% Perforin, and 80% GNYL of native expression levels in the population of CAR-T cells which are not adapted to the hypoxic environment and the hyperbaric environment. In some embodiments, the CAR-T cell therapy comprises an increased expression of HIF1α, TRAIL, FasL, GNLY, GZMM, or combinations thereof. The CAR-T cell therapy of claim 71, wherein the CAR-T cell therapy comprises a decreased expression of HIF2α, GZMB, FasL, Perform, or combinations thereof. In some embodiments, the CAR-T cell therapy comprises increased expression of HIF1α, TRAIL, FasL, GNLY, GZMM, or combinations thereof in amounts of up to 925% HIF1α, 150% TRAIL, 135% FasL, 500% GNLY, and 225% GZMM of native expression levels in the population of CAR-T cells which are not adapted to the hypoxic environment and the hyperbaric environment. In some embodiments, the CAR-T cell therapy comprises increased expression of HIF1α, TRAIL, FasL, GNLY, GZMM, or combinations thereof in amounts of at least 925% HIF1α, 150% TRAIL, 135% FasL, 500% GNLY, and 225% GZMM of native expression levels in the population of CAR-T cells which are not adapted to the hypoxic environment and the hyperbaric environment. In some embodiments, the CAR-T cell therapy comprises a decreased expression of FasL, Perform, GNYL, GZMM, or combinations thereof in amounts up to 80% HIF2α, 30% Perform, 500% GNYL and 220% GZMM of native expression levels in the population of CAR-T cells which are not adapted to the hypoxic environment and the hyperbaric environment. In some embodiments, the CAR-T cell therapy comprises a decreased expression of FasL, Perform, GNYL, GZMM, or combinations thereof in amounts of at least 80% HIF2α, 30% Perform, 500% GNYL and 220% GZMM of native expression levels in the population of CAR-T cells which are not adapted to the hypoxic environment and the hyperbaric environment. In some embodiments, the CAR-T cell therapy comprises a ROR1 CAR. In some embodiments, the CAR-T cell therapy comprises a CD19 CAR. In some embodiments, the CAR-T cell therapy comprises a BCMA CAR. In some embodiments, the CAR-T cell therapy comprises a CD5 CAR. In some embodiments, the CAR-T cell therapy comprises a TRIS CAR. In some embodiments, the CAR-T cell therapy comprises a CD7 CAR. In some embodiments, the CAR-T cell therapy comprises a GPC2 CAR. In some embodiments, the CAR-T cell therapy comprises a NKG2D CAR. In some embodiments, the CAR-T cell therapy comprises a CD14 CAR. In some embodiments, the CAR-T cell therapy comprises an αPD1-MSLN-CAR. In some embodiments, the CAR-T cell therapy comprises a CD33 CAR. In some embodiments, the CAR-T cell therapy comprises a Senl-h19 CAR. In some embodiments, the CAR-T cell therapy comprises an EGFR CAR. In some embodiments, the CAR-T cell therapy comprises an EGFR806 CAR. In some embodiments, the CAR-T cell therapy comprises a GPC3 CAR. In some embodiments, the CAR-T cell therapy has been cultured under the hypobaric environment and the hypoxic environment for at least 50 generations. In some embodiments, the CAR-T cell therapy has been cultured under the hypobaric environment and the hypoxic environment for at least 25 generations. In some embodiments, the CAR-T cell therapy has been cultured under the hypobaric environment and the hypoxic environment for at least 15 generations. In some embodiments, the CAR-T cell therapy has been cultured under the hypobaric environment and the hypoxic environment for at least 10 generations. In some embodiments, the CAR-T cell therapy has been cultured under the hypobaric environment and the hypoxic environment for at least 5 generations. In some embodiments, the CAR-T cell therapy comprises increased cytolytic activity towards cancer cells comprises inducing cytolysis of at least 75% of cancer cells which contact the CAR-T cell therapy. In some embodiments, the CAR-T cell therapy comprises increased cytolytic activity towards cancer cells comprises inducing cytolysis of at least 85% of cancer cells which contact the CAR-T cell therapy.

A first embodiment of the invention includes a method of developing a derivative cell population from a parent cell population, wherein the derivative cell population expresses a phenotype adapted to a hypoxic and hyperbaric environment. Embodiments of the method include culturing a population of the parent cell population under the hypoxic and a hyperbaric condition in vitro for a sufficient time to permit a phenotypically adapted derivative cell population to emerge. A hypoxic and a hyperbaric condition may be referred to as a tumor microenvironment (TME) condition. In some embodiments, a phenotypically adapted cell population may include cells that have been epigenetically reprogrammed. “Sufficient time” for a derivative population to emerge refers to time in serial cell culture, during which time a sufficient number of cell generations have transpired.

In some embodiments, a “cell population” may be highly homogeneous, and understood to be a “cell line” that shows a high degree of phenotypic stability. In some embodiments, the phenotypically adapted derivative cell population comprises an epigenetic alteration that underlies an expression of phenotypic features that distinguish the derivative cell population from the parent cell population. In some embodiments, the phenotypically adapted derivative cell population comprises a genomic alteration that underlies an expression of phenotypic features that distinguish the derivative cell population from the parent cell population.

The phenotypically adapted derivative cell population expresses phenotypic features that distinguish it from the parent cell population. The phenotypic features of the derivative cell line (as particularly expressed under the hypoxic and hyperbaric condition) that distinguish it from the phenotypic features of the parent cell population (i.e., a population without a history of in vitro exposure to the hypoxic and a hyperbaric condition) include one or more of (a) an increased growth rate, (b) an increased viability, (c) an altered metabolic activity profile, or (d) an altered differentiated functionality particular to the parent cell population. By way of example, an altered functionality of an immune cell population may include any cellular behavior that enhances or improves immunologic effectiveness of the derivative cells against target cells, either in vitro or in vivo, as within a solid tumor of a cancer patient being treated with the derivative cell population.

For clarity, noting that a derivative cell population considered to have promise for a human therapeutic application need not show all of the enumerated (a-d) features. And it is also a possibility that the advantage of (d) an elevated immunologic function could outweigh features that may not necessarily seen as advantageous, such as a lower growth rate.

In some of these embodiments, the parent cell population includes an effector immune cell population. In some of these embodiments, the immune cell population includes a natural killer T cell population or a derivative thereof. And in some of these embodiments, the effector immune cell population is derived from a human patient. In some of the embodiments, the effector cell is an immune cell population that specifically targets patient-specific cancer cells residing in a solid tumor.

In some embodiments of the method, the distinguishing phenotypic features of the derivative cell population that permit the phenotypically adapted derivative cell population to emerge include any one or more of (a) transcriptional control, (b) translational control, or (c) mRNA or protein degradation rate.

The distinguishing phenotypic features of the derivative cell population may include an elevated level of a hypoxia response element activation within a hypoxia-induced factor (HIF) gene. Distinguishing phenotypic features of the derivative cell population may include an elevated expression of a hypoxia-induced factor. By way of example, a hypoxia-induced factor may include any one or more of HIF-1a, HIF-1b, HIF-2a, HIF-2b, HIF-1a, or HIF-1b.

In other embodiments, the expression of phenotypic features in the derivative cell population may be mediated by factors other than the HIF factors, such as a member of the Piezo protein family (mechanosensitive ion channel components). In another example, solute carrier (SLC) proteins that act as anion exchange based membrane transporters may become factors that mediate hypoxia effects or responses.

In some embodiments of the method, the parent cell population includes an immune cell population having an immune-system functionality, and wherein, in comparison to the immune functionality of the parent cell population, the derivative cell population includes an elevated level of the immune functionality against a target cell population. By way of a clinical application as a related method, when infused into a patient with a solid tumor that includes the target cell population, the elevated level of immune functionality of the derivative cell population may deliver an effective therapeutic attack against the target cell population.

In some embodiments of the method, the altered level of immunologic functionality of the derivative cell population may include any one or more of an increased cytolytic capability or effectiveness against a target cell population or a target cell apoptotic trigger. By way of a clinical application as a related method, the target cell population may include a resident cancer cell population within a primary solid tumor or in the form of a dispersed population of cells that have metastasized from the primary tumor.

In alternative embodiments of the method, the altered level of immunologic functionality of the derivative cell population includes an increased level of an immunosuppressive effect on a target cell population.

In some embodiments of the method, the distinguishing phenotypic features of the derivative cell population, in comparison to the parent cell population, are expressed under hypoxic and hyperbaric conditions in an in vitro environment. In some embodiments of the method, the distinguishing phenotypic features of the derivative cell population, in comparison to the parent cell population, are expressed under hypoxic and hyperbaric conditions in an in vivo environment. In some embodiments of the method, the in vivo environment includes a tumor site of a patient into which the derivative cell population has been therapeutically infused.

In some embodiments of the method, the parent cell population includes a lymphoid cell developmental lineage. In embodiments, the parent cell population includes a T cell developmental lineage. In embodiments, the parent cell population includes a natural killer (NK) cell population or a derivative thereof. In some embodiments, the parent cell population is a human-derived cell population.

In some embodiments of the method, allowing emergence of a hyperbaric and hypoxic condition-adapted derivative cell population occurs in the absence of a concurrent transfective procedure during the cell culture duration. In other embodiments, the modulation of the phenotype of the parent population to the phenotype of the parent-derivative population occurs in conjunction with a concurrent transfective procedure during the cell culture duration. In still other embodiments, the parent cell population is transfected prior to exposure to culturing under hypoxic and/or hyperbaric conditions.

In some embodiments of the method, after the phenotypically adapted cell derivative population has emerged from the parent population, the method further includes cryogenically preserving a quantity of the derivative cell population as a cell bank in cryogenic vials.

In some embodiments of the method, the method further includes thawing one or more of the cryogenic vials, culturing the thawed cells, and testing the thawed cells to determine whether the hypoxia and hyperbaric adaptations are substantially identical to the adaptations of the cell population prior to cryogenic preservation.

A second embodiment of the invention includes a method of developing a derivative call population from a parent cell population, wherein the derivative cell population expresses a phenotype adapted to a hypoxic environment. This method embodiment includes culturing a population of the parent cell population under a hypoxic condition in vitro for a sufficient number of generations or time to permit a phenotypically adapted derivative cell population to emerge during a cell culture duration.

The phenotypically adapted derivative cell population expresses phenotypic features that distinguish it from the parent cell population. The phenotypic features of the derivative cell population, as expressed under the hypoxic and hyperbaric condition, that distinguish it from the phenotypic features of the parent cell population (without a history of exposure to the hypoxic and a hyperbaric condition) include one or more of (a) an increased growth rate, (b) an increased viability, (c) an altered metabolic activity profile, or (d) an altered differentiated functionality particular to the parent cell population.

For clarity, this second embodiment of the method is directed to hypoxic conditions in vitro, without reference to presence or absence of hyperbaric conditions. All features enumerated in the context of the first method embodiment may be included in the scope of this second embodiment.

A third embodiment of the invention includes a method of developing a derivative call population from a parent cell population, wherein the derivative cell population expresses a phenotype adapted to a hyperbaric environment. This method embodiment includes culturing a population of the parent cell population under a hyperbaric condition in vitro for a sufficient number of generations or time to permit a phenotypically adapted derivative cell population to emerge during a cell culture duration,

The phenotypically adapted derivative cell population expresses phenotypic features that distinguish it from the parent cell population. The phenotypic features of the derivative cell population, as expressed under the hypoxic and hyperbaric condition, that distinguish it from the phenotypic features of the parent cell population (without a history of exposure to the hypoxic and a hyperbaric condition) include one or more of (a) an increased growth rate, (b) an increased viability, (c) an altered metabolic activity profile, or (d) an altered differentiated functionality particular to the parent cell population.

For clarity, this third embodiment of the method is directed to hyperbaric conditions in vitro, without reference to presence or absence of hypoxic conditions. All features enumerated in the context of the first method embodiment may be included in the scope of this second embodiment.

A fourth embodiment of the invention includes a method of determining a level of cytolytic effectiveness of an effector cell population against a target cell population. Cytolytic effectiveness may be understood as a level of immunogenic potency, as measured by a direct or indirect assay of cytolysis against a target cell, typically a cancer cell. This embodiment of the method includes: culturing the target cell population in vitro; and culturing the effector cell population in vitro, wherein the effector cell population has been phenotypically adapted with respect to a parent cell population from which it was derived, wherein the adapted phenotype includes an adaptation to a hyperbaric and hypoxic environment. The method embodiment continues by combining a portion of the cultured target cell population and a portion of the cultured effector cell population together to form a combined culture; culturing the combined target cell population and the effector cell population together for a culture duration; and determining the level of cytolysis of the target cell population as a consequence of the presence of the effector cell population.

In some embodiments of the fourth embodiment, the effector cell population includes natural killer T cell or a derivative thereof. In some embodiments, the target cell population includes a cancer cell population. In some embodiments, the cancer cell population is sourced from a human cancer patient. In some embodiments, the target cell population includes a population of cancer cells resident within a solid tumor.

Some embodiments of the method are suitable as a companion diagnostic (CDx) assay that measures a level of cytolytic effectiveness or immunogenic potency that correlates with a level of therapeutic effectiveness of the effector cell population against a human cancer cell population within a tumor, within a patient.

A fifth embodiment of the invention includes a method of treating cancer in a patient includes providing an effector cell population that has, in response to being cultured in a hyperbaric and hypoxic condition, has undergone a phenotypic adaptation that supports one or more expressed features that, in comparison to a cell population from which the effector cell population was derived, improve a performance of the effector cell population under hypoxic and/or hyperbaric conditions, or under tumor microenvironment conditions, wherein the performance of the effector cell population includes an immunologic functionality against a target cell population. The method embodiment may further include infusing a physiologically effective amount of the effector cell population into a patient to treat a tumor includes the target cell.

A sixth embodiment of the invention includes a cell population as a product that is adapted to a hypoxic and hyperbaric condition. Some embodiments are in the form of an immune cell product. Criteria that apply to this product follow.

The adapted cell population is derived from an originating parent cell population not-adapted to a hypoxic and/or a hyperbaric condition; the adapted cell population, in transitioning from the originating parent cell population, has been cultured for a sufficient number of generations under a hypoxic and a hyperbaric condition to permit a phenotypic adaptation to occur that underlies adaptation to the hypoxic and a hyperbaric condition. In one embodiment, a sufficient number of generations may include 5, 10, 15, or 20 generations.

The phenotypically adapted derivative cell population has phenotypic features that distinguish it from the parent cell population, and the phenotypic features of the derivative cell population, as expressed under the hypoxic and hyperbaric condition, that distinguish it from the phenotypic features of the parent cell population (without a history of exposure to the hypoxic and a hyperbaric condition) includes one or more of (a) an increased growth rate, (b) an increased viability, (c) an altered metabolic activity profile, or (d) an altered differentiated functionality particular to the parent cell population.

A seventh embodiment of the invention includes a method cell banking that includes freezing a cell population of interest in a liquid cryopreserving medium to form a frozen cell-banked cell population; thawing the frozen cell population into a liquid cell culture medium; culturing the thawed cell population in a cell culture system that can regulate (a) a total gas atmospheric pressure and (b) a hypoxic gas composition to form a hyperbaric and hypoxic gas environment that surrounds a cell culture container in the system, wherein the total gas atmospheric pressure and the hypoxic gas composition are regulated independently of each other. The method may further include expanding the cell population within the cell culture system, and within the hyperbaric and hypoxic gas environment, to yield a cell population expanded to a desired level.

In embodiments, prior to freezing the cell population of interest, the method includes culturing the population in the hyperbaric and hypoxic gas environment provided by the cell culture system.

In other embodiments, the cell population of interest is a population derived from a parent population, the cell population of interest being phenotypically distinct from the parent population. Distinctness from the parent population may include any one or more of (a) an increased growth rate, (b) an increased viability, (c) an altered metabolic activity profile, (d) an altered morphology, or (e) an altered functional feature with respect to that of the parent cell population.

An eighth embodiment of the invention includes a pharmaceutical composition that includes a cell population suitable for infusion into a patient, the cell population having an adaptation to a hyperbaric and hypoxic condition wherein the adapted cell population is derived from an originating parent cell population not-adapted to a hypoxic and/or a hyperbaric condition. With reference to the cell population included within the pharmaceutical composition, the follow criteria apply.

The adapted cell population, in transitioning from the originating parent cell population, has been cultured for a sufficient number of generations under a hypoxic and a hyperbaric condition to permit a phenotypic adaptation to occur that underlies adaptation to the hypoxic and a hyperbaric condition; the phenotypically adapted derivative cell population includes phenotypic features that distinguish it from the parent cell population. Further, the phenotypic features of the derivative cell population, as expressed under the hypoxic and hyperbaric condition, that distinguish it from the phenotypic features of the parent cell population (without a history of exposure to the hypoxic and a hyperbaric condition) includes one or more of (a) an increased growth rate, (b) an increased viability, (c) an altered metabolic activity profile, or (d) an altered differentiated functionality particular to the parent cell population.

A ninth embodiment of the invention includes a modified immune cell population derived from a native immune cell population, modified for increased cytolytic activity or immunogenic potency towards a cancer cell population relative to the native immune cell population, the modified immune cell population comprising: a decreased expression of a first protein, wherein the first protein comprises HIF2α, granzyme B (GZMB), perform, FasL, or GNYL; and an increased expression of a second protein, wherein the protein comprises HIF1α, TRAIL, FasL, GNLY, or GZMM.

In some embodiments, the modified immune cell population does not differ from the native immune cell population with respect to expression of beta-actin. In some embodiments, a decreased expression of granzyme B, wherein the decreased expression of granzyme B comprises a 5, 10, 15, 20, 25, 30, 35, 40, 45, 50, 55, 60, 65, 70, 75, 80, 85, 90, or 95% of a native level of granzyme B expression. In some embodiments, a decreased expression of perform, wherein the decreased expression of perform comprises a 5, 10, 15, 20, 25, 30, 35, 40, 45, 50, 55, 60, 65, 70, 75, 80, 85, 90, or 95% of a native level of perforin expression. In some embodiments, an increased expression of TRAIL, wherein the increased expression of TRAIL comprises a 5, 10, 15, 20, 25, 30, 35, 40, 45, 50, 55, 60, 65, 70, or 75% increase in a native level TRAIL expression. In some embodiments, an increased expression of HIF1α, wherein the increased expression of HIF1α comprises a 200, 250, 300, 350, 400, 450, 500, 600, 700, 800, 900, or 1000% increase in a native level HIF1α expression. In some embodiments, a decreased expression of HIF2α, wherein the decreased expression of HIF2a comprises a 10, 20, 30, 40, 50, 60, 70, 80, 90, or 100% of a native level of HIF2a expression. In some embodiments, a decreased expression of FASL, wherein the decrease in expression of FASL comprises 5, 10, 15, 20, 25, 30, 35, 40, 45, 50, 55, 60, 65, 70, 75, 80, 85, 90, or 95% of a native level of FASL expression. In some embodiments, an increased expression of FasL, wherein the increased expression of FasL comprises a 10, 20, 30, 40, 50, 60, 70, 80, 90, or 100, 110, 120, 125, 130, 140, 150, 160, 170, 180, 190, or 200% increase in a native level FasL expression. In some embodiments, a decreased expression of GNLY, wherein the decrease in expression of GNLY comprises 5, 10, 15, 20, 25, 30, 35, 40, 45, 50, 55, 60, 65, 70, 75, 80, 85, 90, or 95% of a native level of GNLY expression. In some embodiments, an increased expression of GNLY, wherein the increased expression of GNLY comprises 50, 60, 70, 80, 90, or 100, 110, 120, 125, 130, 140, 150, 160, 170, 180, 190, 200, 250, 300, 350, 400, 450, or 500% increase in a native level GNLY expression. In some embodiments, an increased expression of GZMM, wherein the increased expression of GZMM comprises 5, 10, 15, 20, 25, 30, 35, 40, 45, 50, 60, 70, 80, 90, or 100, 110, 120, 125, 130, 140, 150, 160, 170, 180, 190, 200, 210, 220, 230, 240, 250, 260, 270, 280, 290, 300, 325, 350, 375, or 400% increase in a native level GZMM expression.

In some embodiments, the modified immune cell population comprises T cells, or wherein the native immune cell population comprises T cells. In some embodiments, the modified immune cell population comprises natural killer T cells, or wherein the native immune cell population comprises natural killer T cells. In some embodiments, the modified immune cell population comprises NK-92 cells, or wherein the native immune cell population comprises NK-92 cells. In some embodiments, an increased expression of HIF1α, TRAIL, FasL, GNLY, and GZMM. The modified immune cell population of the immediately preceding claim further comprising a decreased expression of HIF2α, GZMB, and Perform. The modified immune cell population of the immediately preceding claim further comprising an increased expression of HIF1α, TRAIL, FasL, GNLY, and GZMM in the approximate amounts of 610% HIF1α, 125% TRAIL, 150% FasL, 300% GNLY, and 300% GZMM of native expression levels. The modified immune cell population of the immediately preceding claim further comprising a decreased expression of HIF2α, GZMB, and Perforin in the approximate amounts of 90% HIF2α, 25% GZMB, and 35% Perform of a native level of expression of native expression levels. In some embodiments, the modified immune cell population is long term adapted to hypoxic conditions. In some embodiments, an increased expression of HIF1α, TRAIL, and GZMM. The modified immune cell population of the immediately preceding claim further comprising a decreased expression of FasL, Perform, and GNYL. The modified immune cell population of the immediately preceding claim an increased expression of HIF1α, TRAIL, and GZMM in the approximate amounts of 290% HIF1α, 150% TRAIL, and 175% GZMM of native expression levels. The modified immune cell population of the immediately preceding claim further comprising a decreased expression of FasL, Perforin, and GNYL in the approximate amounts of, 33% FasL, 50% Perforin, and 80% GNYL of native expression levels. In some embodiments, the modified immune cell population is short term adapted to hypoxic conditions. In some embodiments, an increased expression of HIF1α, TRAIL, FasL, GNLY, and GZMM. The modified immune cell population of the immediately preceding claim further comprising a decreased expression of HIF2α, GZMB, FasL, and Perform. The modified immune cell population of the immediately preceding claim an increased expression of HIF1α, TRAIL, FasL, GNLY, and GZMM in the approximate amounts of increased expression of 925% HIF1α, 150% TRAIL, 135% FasL, 500% GNLY, and 225% GZMM of native expression levels. The modified immune cell population of the immediately preceding claim further comprising a decreased expression of FasL, Perform, GNYL and GZMM in the approximate amounts of 80% HIF2α, 30% Perforin, 500% GNYL and 220% GZMM of native expression levels. In some embodiments, the modified immune cell population is long term adapted to hypoxic and hyperbaric conditions. In some embodiments, an increased expression of HIF1α, TRAIL, and GZMM. The modified immune cell population of the immediately preceding claim further comprising a decreased expression of FasL, GNLY, and Perform. The modified immune cell population of the immediately preceding claim an increased expression of HIF1α, TRAIL, and GZMM in the approximate amounts of increased expression of 525% HIF1α, 125% TRAIL, and 175% GZMM of native expression levels. The modified immune cell population of the immediately preceding claim further comprising a decreased expression of FasL, GNLY, and Perforin in the approximate amounts of 25% FasL, 50% GNLY, and 33% Perform of native expression levels. In some embodiments, the modified immune cell population is short term adapted to hypoxic and hyperbaric conditions. In some embodiments, the modified immune cell population induces cytolysis of cancer cells by death receptor mediated cytotoxicity. In some embodiments, death receptor mediated cytotoxicity is induced by increased expression of FasL or TRAI1. In some embodiments, the modified immune cell population exhibits increased cytolytic activity or immunogenic potency towards cancer cells at ratios 1:1; 2:1; 5:1; and 10:1 of modified immune cells to cancer cells. In some embodiments, the increased cytolytic activity towards cancer cells comprises inducing cytolysis of 5, 10, 15, 20, 25, 30, 35, 40, 45, 50, 55, 60, 65, 70, 75, 80, 85, 90, 95, 99, or 100% of cancer cells in solid tumor.

### BRIEF DESCRIPTION OF THE FIGURES

FIGS. 1A-1D provide data regarding the cytolytic capability or immunogenic potency of a model immune effector cell population (NK-92) and a TME-adapted population derived therefrom, both populations cultured under an ambient cell culture condition and under a TME condition. FIG. 1A shows target cells being exposed to an effector cell population in ambient culture conditions. The effector cells were either the parental cell line (gray) or cells that have been adapted to TME conditions of 1% O2 and 2 PSIG (black). The effector cells were added to target cells at a ratio of 5:1 (effector cells to target cells), and subsequent cytolysis of target cells was measured over time.

FIG. 1B shows target cells being exposed to an effector cell population in TME condition culture conditions. The effector cells were either the parental cell line (gray) or have been adapted to TME conditions of 1% O2 and 2 PSIG (black). The effector cells were added to target cells at a ratio of 5:1 (effector cells to target cells), and subsequent cytolysis of target cells was measured over time. The data shows cytolysis at four time points.

FIG. 1C shows target cells being exposed to an effector cell population in ambient condition culture conditions.

FIG. 1D shows target cells being exposed to an effector cell population under TME conditions. The effector cells were either the parental cell line (gray) or have been adapted to TME conditions of 1% O2 and 2 PSIG (black). The effector cells were added to target cells at a ratio of 5:1 (effector cells to target cells), and subsequent cytolysis of target cells was measured over time. The data show cytolysis at four time points.

FIG. 1E shows the percent difference in the area under the curve between target cell cytolysis over 46 hours measured under TME versus “normoxic”, “ambient” or “standard” conditions, referring to a conventional or standard CO2 incubator. Error bars indicate standard error of mean.

FIGS. 2A-2B provide data from an analysis of the effector-to-target ratio as applied to a cytolytic assay performance of a model immune effector cell population (NK-92) and a TME-adapted population derived therefrom, the data validating the assay. FIG. 2A shows target cells being exposed to an effector cell population under an ambient condition. The effector cells were either the parental cell line (gray) or cells that have been adapted to TME conditions of 1% O2 and 2 PSIG (black). Effector cells added to target cells at various ratios (effector cells to target cells), and subsequent cytolysis of target cells was measured over time. Error bars indicate standard error of mean

FIG. 2B shows target cells being exposed to an effector cell population under a TME condition.

FIGS. 3A-3D provide data from an analysis of the maintenance of the cytolytic performance of a model immune effector cell population (NK-92) and a TME-adapted population derived therefrom over repeated stimulation.

FIG. 3A shows effector cell populations being repeatedly exposed to target cells under an ambient condition. The effector cells were either the parental cell line (gray) or cells that have been adapted to TME conditions of 1% O2 and 2 PSIG (black). The effector cells were added to target cells at a ratio of 5:1 (effector cells to target cells), and subsequent cytolysis of target cells was measured over time. After 24 hours, effector cells were counted and transferred to a well with fresh target cells, maintaining a ratio of 5:1 (effector cells to target cells), and subsequent cytolysis of target cells was measured over time. This was repeated two more times for a total of four stimulations.

FIG. 3B shows effector cell populations being repeatedly exposed to target cells under TME conditions.

FIGS. 3C-D show immune effector cell populations being repeatedly exposed to target cells under both ambient and TME conditions. FIG. 3C shows immune effector cell populations being repeatedly exposed to target cells under ambient conditions. The data show cytolysis at 24 hours after coculture of effector and target cells. Error bars indicate standard error of mean.

FIG. 3D shows effector cell populations being repeatedly exposed to target cells under TME conditions.

FIGS. 4A-4C provide date of an RNAseq analysis of gene expression in a model immune effector cell population (NK-92) and in a TME-adapted population derived therefrom.

FIG. 4A shows an RNAseq analysis of Log 2 Fold change in the expression of 19 cytotoxic genes in adapted cells relative to parental cells.

FIG. 4B shows a Western Blot analysis of protein expression in parental cells and adapted cells; proteins include HIF1α, FasL, Granzyme B, Perforin, and Actin.

FIG. 4C shows density analysis of western blot, indicating relative expression of indicate proteins in adapted cells relative to parental cells. Dotted line indicates equivalent expression to parental.

FIGS. 5A-5E provide data related to the cytolytic capability of a model immune effector cell population (CAR-T) and a TME-adapted population derived therefrom, both populations cultured under an ambient cell culture condition and under a TME condition, and then assayed under both an ambient culture condition and a TME condition.

FIG. 5A shows target cells being exposed to an effector cell population under an ambient condition. The effector cells were either CAR-T cells grown under ambient conditions (gray) or cells that have been adapted to TME conditions of Ambient O2 and 4 PSIG (black). The effector cells were added to target cells at a ratio of 10:1 (effector cells to target cells), and subsequent cytolysis of target cells was measured over time.

FIG. 5B shows target cells being exposed to an effector cell population under a TME condition.

FIGS. 5C-5D show target cells being exposed to an effector cell population under both ambient and TME conditions. FIG. 5C shows target cells being exposed to an effector cell population under an ambient condition. The effector cells were either CAR-T cells grown under ambient conditions (gray) or cells that have been adapted to TME conditions of Ambient O2 and 4 PSIG (black). The effector cells were added to target cells at a ratio of 10:1 (effector cells to target cells), and subsequent cytolysis of target cells was measured over time. The data shows cytolysis at two time points.

FIG. 5D shows target cells being exposed to an effector cell population under a TME condition.

FIG. 5E shows effector cell populations being repeatedly exposed to target cells under ambient conditions. The effector cells were either CAR-T cells grown under ambient conditions (gray) or cells that have been adapted to TME conditions of 3% O2 and 4 PSIG (black). The effector cells were added to target cells at a ratio of 10:1 (effector cells to target cells), and subsequent cytolysis of target cells was measured over time. After 48 hours, effector cells were counted and transferred to a well with fresh target cells, maintaining a ratio of 10:1 (effector cells to target cells), and subsequent cytolysis of target cells was measured over time. This was repeated two more times for a total of four stimulations.

### DETAILED DESCRIPTION

The natural tumor microenvironment (TME) of solid tumors has several features that differ from a normal tissue environment, including elevated interstitial fluid pressure and a relative hypoxia. These features are considered a consequence of a number of factors, including tumor tissue organization and vascularity. The hyperbaric and hypoxic aspect of a tumor condition can influence the gene and protein expression within tumor cells, and thus modulate the evident phenotype of tumors as a whole. This disclosure relates to phenotypic differences between a parent effector immune cell population and a derivative population, these differences arising, at least in part, because of exposure to a cell culture environment that includes (relative to an ambient atmospheric condition) hypoxia and/or a hyperbaric atmosphere.

The modulation of phenotypic features within a cell population is broadly understood as a phenotypic plasticity. The efficacy of treatment with anti-cancer therapeutic agents (small molecules, protein biologics, and immune cells) is also subject to the hyperbaric and hypoxic conditions of the tumor microenvironment, as well as the overall efficiency of the vasculature of the tumor. In general, the TME conditions (poor vascularity, hypoxia, elevated pressure) within a tumor is not conducive to therapeutic intervention via the vasculature, and is particularly detrimental to the success of cell-based therapeutics. In some instances, the expressed phenotype of a tumor within a TME may encourage tumor metastasis and anti-cancer drug resistance.

Phenotype generally refers to features of a cell population that are observable in any way. Observable features may include, for example, as cell morphology, dimension, adherent properties, electrical properties, metabolic activity, or migratory behavior. On another level, phenotypic differences can manifest by way of differences in messenger RNA expression of the cellular genome, or rates of protein transcription from expressed RNA. Although the inventors generally believe that phenotypic features described here may not reflect genotypic changes in populations, such a possibility cannot be excluded, and indeed, in some embodiments, genotypic changes in derivative cell populations may occur.

It is appreciated by the inventors that cell therapies which are used as treatments for cancer may function less effectively in vivo than in in vitro models due to the failure of in vitro models or in vitro culture environments under which cell therapies are produced to replicate the in vivo tumor microenvironment under which the cell therapies function. Cell therapies which are administered in vivo may thus function less effectively than expected based on in vitro models, or may require an extended adaptation period once they are administered in vivo as to adapt to the tumor microenvironment under which they are expected to function, often to the detriment of the patient who will not obtain the full benefit of the cell therapy during the adaptation period. Accordingly, disclosed herein are methods of preparing cell therapies under tumor microenvironment (TME) conditions, for example, hypoxic and hyperbaric conditions, for increased cytotoxicity towards cancer cells. Also disclosed herein are improved cell therapies with increased cytotoxicity towards cancer cells.

### Approach, Methods, and Results

### NK-92 Model

To address the therapeutic limitations of an immune effector cell within a tumor microenvironment, the development of a derivative effector cell population with improved functionality in a TME environment was sought. NK-92 cell lines were serially passaged and maintained under tumor microenvironment culture conditions (2.0 PSI+1% O2) for a minimum of five weeks. The result of this extended culture duration was to precondition and expand a derivative population of NK-92 cells (TME-adapted NK-92 cells) with a level of oxygen dependency lower than that of the parent NK-92 cell line. This lower level of oxygen dependency could be the result of a widespread phenotypic modification within the derivative population, or a consequence of enriching for a subset of cells that have metabolically adapted to pressurized and hypoxic culture condition. In some embodiments, phenotypic modification may be understood as a reprogramming of gene expression, an epigenetic process that leaves the underlying genome intact. In some embodiments, a derivative population may include genotypic changes developed during the extended time in serial passage culture.

The derivative population of TME-adapted NK-92 cells that was maintained and expanded under TME culture conditions initially showed a decreased growth rate compared to a parent NK-92 cell population maintained under ambient oxygen (21% O2) culture conditions, but the derivative population gradually increased its growth rate to reach parity with the growth rate of the parent cohort.

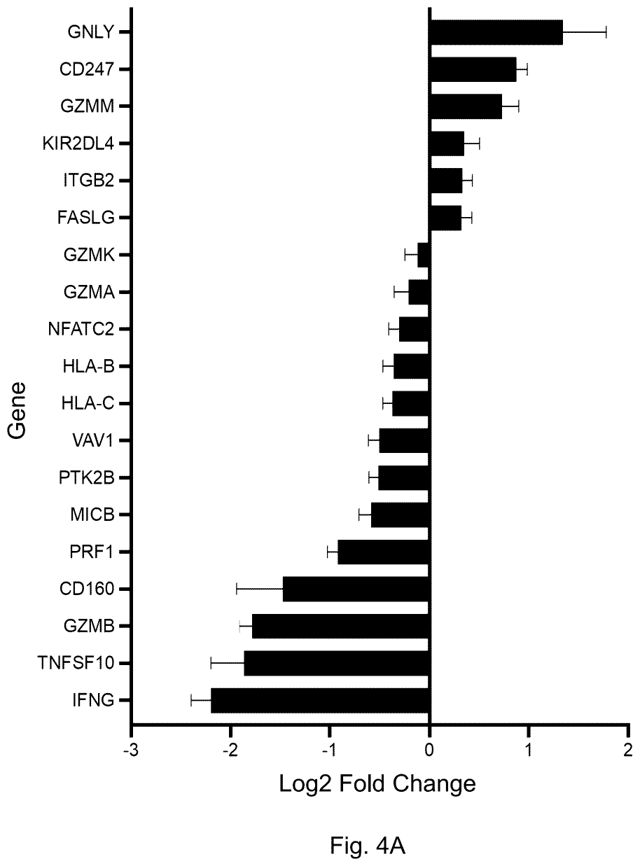

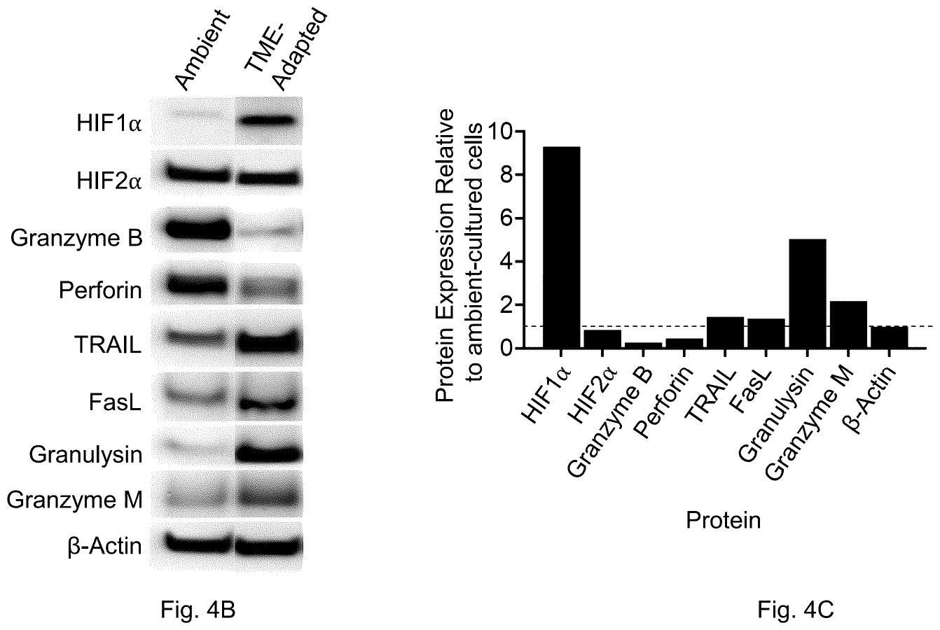

This period of TME adaptation to the TME condition (for example, five weeks) in terms of growth rate, coincided with an enhancement of the tumor killing properties for TME-Adapted NK-92 cell derivative cell population against A549 cells, a non-small cell lung cancer cell line. Enhanced cytolytic activity or immunogenic potency of TME-Adapted NK-92 cells was about two-fold greater than their non-TME adapted parent population counterparts, with sustained potency observed in both ambient and TME culture conditions. Further, serial exhaustion studies, in which TME-Adapted NK-92 cells were repeatedly stimulated with tumor cells, show a sustained level of cytolytic activity, in contrast to non-TME adapted NK92 counterparts. TME-Adapted NK-92 cells also exhibit lower levels of key cytolytic agents granzyme B and perforin expression (FIGS. 4B-4C), as assessed by flow cytometry, while exhibiting distinct gene expression signatures as profiled with RNA sequence analysis (RNAseq). This latter observation suggests that the mechanism by which TME-adaption occurs is not by way of the action of granzyme B and perform, but rather possibly by way of elevated activity of cell surface receptors for FasL or Trail, as discussed below, from the perspective of Prager and Watzel (J Leukocyte Bio, Vol: 105, No: 6, pp 1319-1329, 20 May 2019).

### Primary Human T Cell Models

A process similar to the TME adaptation of NK-92 cells, as described above, can also be performed with primary cells to yield a TME-adapted derivative cell population. To this end, primary human T cells were transduced with a lentiviral encoding the ROR1 chimeric antigen receptor (CAR), redirecting the specificity of these T cells to ROR1-expressing (tyrosine kinase ROR1) tumor cell lines. ROR1-CAR-T cells were expanded under ambient oxygen (21% O2) and pressure (0 PSIG), or TME culture conditions for nine days. These cells exhibited enhanced tumor killing properties against SKOV3 cells, an ovarian cancer cell line that expresses ROR1. Enhanced cytolytic activity of TME-adapted ROR1-CAR-T cells was about 10-20% greater than their non-TME adapted counterparts, with sustained potency observed in both ambient and TME culture condition. Further, serial exhaustion studies, in which ROR1-CAR-T cells were repeatedly stimulated with tumor cells, show a sustained level of cytolytic activity, in contrast to non-TME adapted ROR1-CAR-T cells.

These data support the hypothesis that immune cells can both react and functionally adapt to different microenvironments. TME-adaptation strategies during cell expansion can enhance potency and efficacy of cell therapies designed to work in a solid tumor microenvironment.

Although the data disclosed herein relate to immune effector cell populations and their functionalities, the same or similar approach of long term serial culture on target cell populations, or on populations of any type of cell, may have similar phenotypically adaptive consequences.

### Cell Density, and Impedance as a Surrogate for Cell Density

Cell density generally refers to the number of cells in a cell culture environment per unit cell culture container volume. Alternatively, for cells that attach to a cell culture container surface, cell density may refer the number of cells per unit surface area. The cell culture populations addressed in this application are typically attached to a culture surface, and thus cell density can be expressed either as cells/unit culture volume or cells/unit surface area.

Cell culture performance is often expressed in volumetric terms, for example the level of a product or a metabolite per unit cell culture volume. Including cell density in the performance terminology allows data to be further expressed from a cell-based perspective. For example, the level of a product or metabolite can be expressed as a level of production or metabolism per unit cell. Data such as these can be used for cell culture performance monitoring, process analysis, and for feedback control of a cell culture process.

In some cell culture contexts, it is advantageous for cell density measurements to be taken non-invasively of the culture or without conveying a sample external to the culture. Thus, optical data can used to capture cells/unit volume as well as cell sizing. The amount of optical equipment and its footprint can be a burden when a cell culture study includes many replicates and groups. The use of impedance as a monitoring tool provides advantages by lowering the level of the equipment footprint, while providing high-resolution data in real time.

Cell culture-monitoring impedance measurements rely on an electrical grid embedded in the lower surface of a cell culture container. Multi-well cell culture plates are appropriate for studies, the culture containers within the plates typically referred to as wells. Multi-well plates are provided in different sizes, are compact and stackable, and can accommodate multiple replicates, numbers of groups, and standard curve wells.

Impedance across a cell culture surface is measured as reactance; as cell culture data, the reactance value is a surrogate for cell density. For use as a cell culture process parameter, reactance data can be used directly, without conversion into a formal cell density value. However, conversion to a cell density value is easily done by considering cell population specifics, such as cell size, the degree to which cells within the culture spread when growing or compact themselves at higher densities. Another cell population variable is the degree to which cells remain as a single layer, or if they grow over each other.

All of these considerations are resolvable when working with a known cell and its characteristics in culture, and by making use of a standard curve, wherein a known level of cells are distributed into standard wells, along with cells distributed into experimental wells. Individual cell populations can vary in their features that contribute to a cell-based impedance signal, thus the standard curve and how to apply its analysis to determining the cell density in test wells is a focus of attention.

As described below, one assay of effector cell ability to kill target cells is to co-culture them for a period in the same culture wells. In experimental details below, effector cells (NK-92 and derivatives thereof) are added to ongoing cultures of target cells (A549). In these assays, the cells of interest in the co-culture with respect to impedance data are the target cells. The presence of the effector cells in the co-culture does not significantly affect the impedance readings, which thus remain a reliable surrogate for viable cell density. This follows from the target cell characteristic of strong attachment to the cell culture surface. While the effector cells settle to the bottom of the cell culture well, they do not attach and spread on the surface.

### Experimental Results

The “effector cells” used as models in the studies described herein includes populations of the natural killer T-cell, NK-92, and cell populations derivative (and distinct from the parent NK-92) which have been serially-passaged through tumor microenvironment (TME) conditions, as well as ROR1-CAR-T cells, which have been expanded in TME conditions for 9 days. NK-92 cells were provided by the ATCC (CRL-2407) and used herein as a research model.

ROR1-CAR-T cells were generated via lentiviral transduction. ROR1 CAR-encoding lentivirus was purchased from Promab Biotechnologies (Richmond CA). The “target cell population” used in the studies described herein is the non-small cell lung cancer line, A549, and ovarian cancer cell line, SKOV3, as provided by the ATCC (CCL-185 and HTB-77, respectively).

Ambient conditions, as used herein, refers to cells being cultured in a conventional CO2 incubator wherein the atmospheric pressure is the same as the ambient atmospheric pressure external to the incubator: 101,325 Pa, or, in other terms, equivalent to 1013.25 millibars, 760 mm Hg, 29.9212 inches Hg, or 14.696 psi. Ambient oxygen level (at sea level) is typically described as a percent of the total atmospheric composition, or 20.9%. Within a conventional CO2 incubator, the actual O2 level is decreased slightly by the addition of CO2 (5%-10%).

Relative to ambient conditions, TME conditions are hypoxic and hyperbaric, as provided by the Avatar instrument (Xcell Biosciences, Inc., San Francisco, CA). The hypoxic O2 level used experimentally is about 1%. The hyperbaric condition is 2 PSIG, this representing 2 PSIG (i.e., 2 PSI above the ambient pressure). The experimental set ups are detailed in Tables 1-3, below.

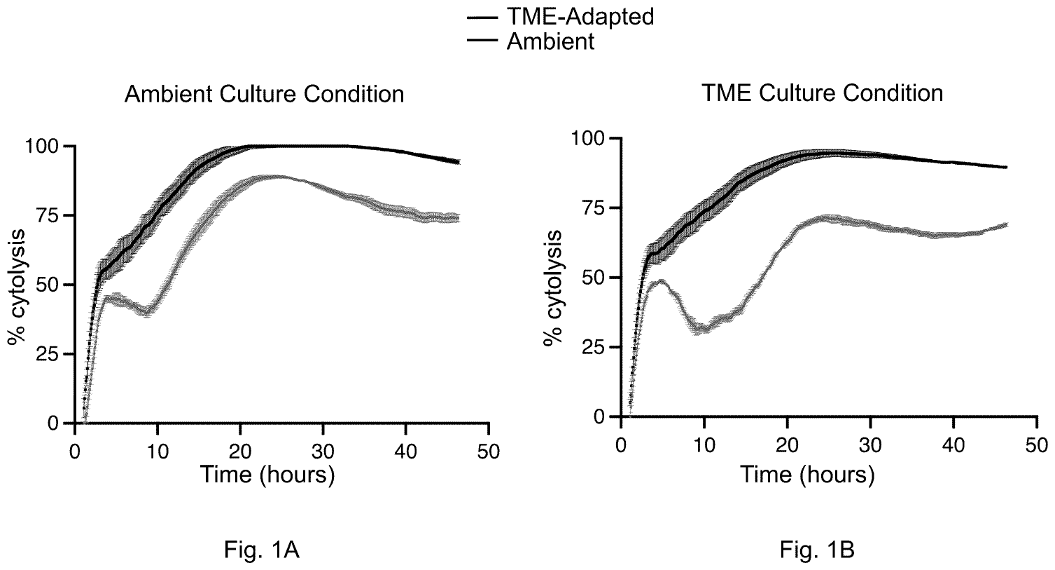

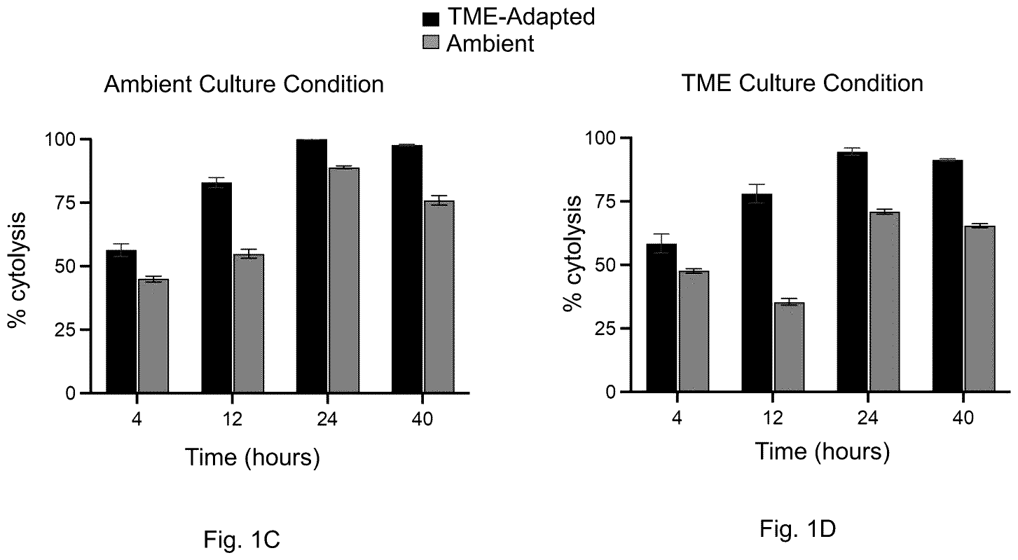

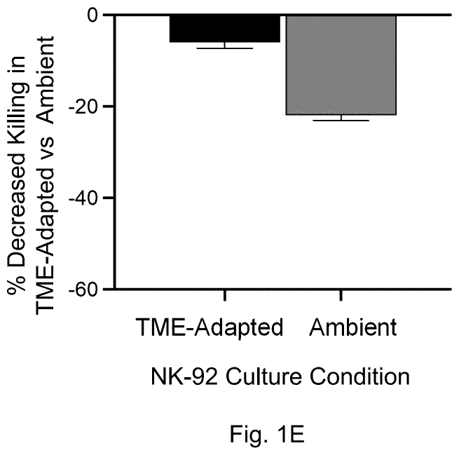

FIGS. 1A-1B show target cells being exposed to an effector cell population under both ambient (FIG. 1A) and TME (FIG. 1B) conditions. The effector cells were either the parental cell line or cells that have been adapted to TME conditions of 1% O2 and 2 PSIG. These effector cell conditions are identified in Table 1, below. The effector cells were added to target cells at a ratio of 5:1 (effector cells to target cells), and subsequent cytolysis of target cells was measured over time. TME adapted NK-92 cells exhibited increased cytotoxic activity, with faster killing kinetics and higher total cytolysis. When experiments were repeated under TME conditions, the differences between NK-92 culture conditions were even more stark.

FIGS. 1C-1D show this cytolysis data quantified at four times points. This analysis shows that TME-adapted NK-92 cells exhibit significantly increased cytolysis at 4, 12, 24, and 40 hours after initiation of coculture with A549 cells, as compared to parental NK-92 cells. This increased cytolysis is observed when coculture occurs at either ambient or TME conditions.

FIG. 1E shows the percent difference in the area under the curve between target cell cytolysis over 46 hours measured under TME versus normoxic (ambient) conditions. While parental NK-92 cells exhibited an average 21% reduction in killing activity under TME conditions, TME adapted NK-92 cells exhibited only slightly reduced killing in TME conditions, with an average 6% reduction relative to ambient.

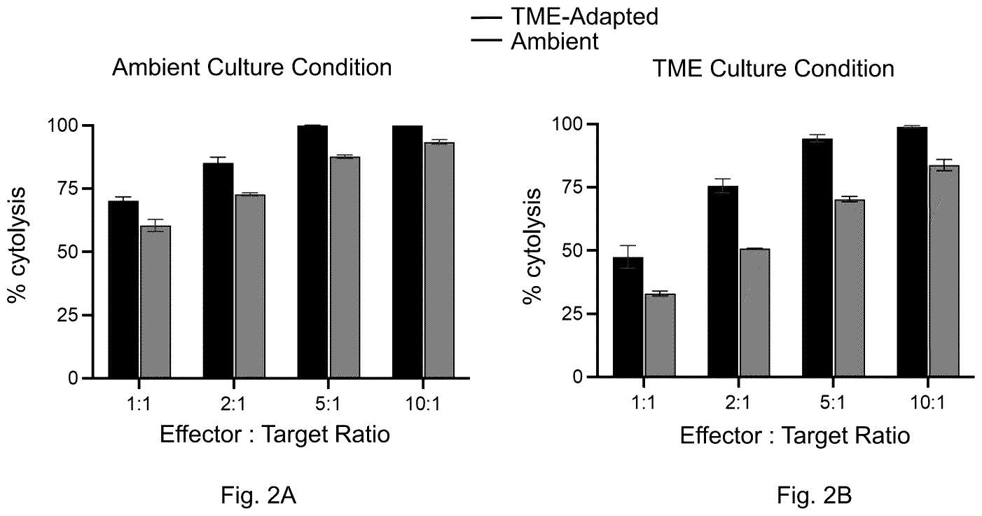

FIGS. 2A-2B show target cells being exposed to an effector cell population under both ambient (FIG. 2A) and TME (FIG. 2B) conditions. The effector cells were either the parental cell line or cells that have been adapted to TME conditions of 1% O2 and 2 PSIG. Effector cells were added to target cells at various ratios (effector cells to target cells), and subsequent cytolysis of target cells was measured over time. The target-to-effector (ET) cell ratios are identified in Table 2, below. Briefly, target cells were consistently seeded at 10,000 cells/well. Effector cells were seeded various at ratios of 0:1 (no effector cell control), 1:1 (10,000 cells/well), 2:1 (20,000 cells/well), 5:1 (50,000 cells/well), and 10:1 (100,000 cells/well).

FIG. 2A shows that effector cells at all concentrations increase target cell cytolysis, with a greater effect being achieved by increasing the effector cell addition. FIG. 2B shows a result similar to that shown FIG. 2A.

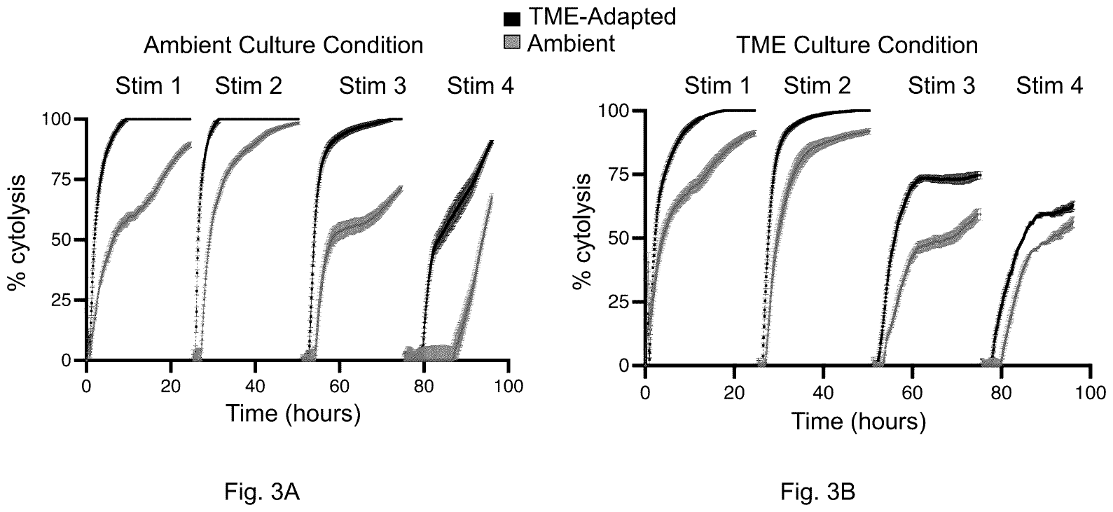

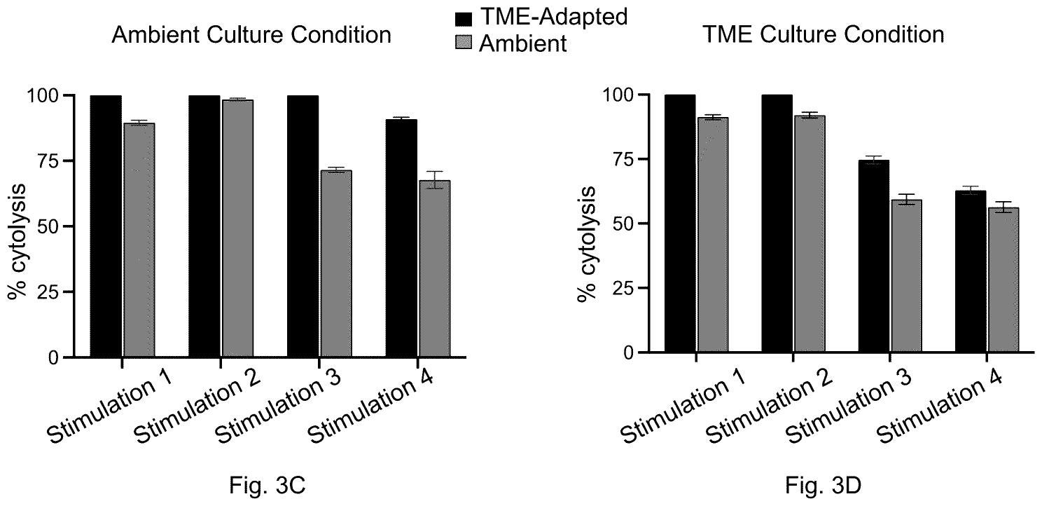

FIGS. 3A-B show effector cell populations being repeatedly exposed to target cells under both ambient (FIG. 3A) and TME (FIG. 3B) conditions, for a total of four stimulations. The effector cells were either the parental cell line or cells that have been adapted to TME conditions of 1% O2 and 2 PSIG. The effector cells were added to target cells at a ratio of 5:1 (effector cells to target cells), and subsequent cytolysis of target cells was measured over time. TME adapted NK-92 cells exhibited increased cytotoxic activity across all four stimulations, with faster killing kinetics and higher total cytolysis.

FIG. 3C-D show this cytolysis data quantified at 24 hours after initiation of each stimulation. This analysis shows that TME-adapted NK-92 cells exhibit significantly increased cytolysis at each stimulation with A549 cells, as compared to parental NK-92 cells. This increased cytolysis is observed when coculture occurs at either ambient or TME conditions.

FIG. 4A shows an RNAseq analysis of Log 2 Fold change in the expression of 19 cytotoxic genes in adapted cells relative to parental cells. Many genes commonly associated with NK cell killing were downregulated in adapted cells, as compared to parental cells. These genes included IFNG, which encodes the cytokine IFN-γ, GZMB, which encodes the serine protease Granzyme B, and PRF1, which encodes the pore-forming protein Perform 1. This was surprising, as adapted cells exhibited higher NK-mediated cytolysis in both normoxic (i.e., ambient level oxygen) and TME conditions. Adapted cells exhibited upregulation of GNLY, which encodes the cytotoxic protein granulysin, GZMM, the serine protease Granzyme M, and FASLG, which encodes the Fas Ligand death receptor.

FIG. 4B shows a Western Blot analysis of protein expression in parental cells and adapted cells; proteins include HIF1α, HIF2α, Granzyme B, Perform, GNLY, TRAIL, FasL, GNLY, GZMM, and Actin. FIG. 4C shows a density analysis of western blot data, indicating relative expression of proteins in adapted cells relative to parental cells. Dotted line indicates equivalent expression to parental. HIF1α (hypoxia-induced factor 1a) shows a 5-fold increase over the parental cells. This HIF1α-affect is a well-known phenomenon and validates the assay as being an understandable and effective reflection of exposure to TME conditions. Actin, in contrast, is not considered to be responsive to TME conditions. Thus, the invariability of actin validates the assay be being consistent with that view, and further validates as showing consistent Western Blot handling of cell culture samples.

Granzyme B and perform are both implicated in the literature as involved in the killing of target cells by natural killer cells. The results of FIGS. 4A-C suggest that neither of these are demonstrably involved in TME-modulation of the ability of NK-92 to kill target cells. In contrast FasL shows at approximate 50% increase in response to TME conditions, thus implicating the FasL or Trail as involved in the TME adaptation response.

Without being bound to a particular theory, and for the context of the invention, possible mechanisms of action of natural killer cell-mediated cellular cytotoxicity as described by Prager and Watzel (referenced above). There is evidence for two distinct natural killer cell killing mechanisms, not mutually exclusive. One mechanism involves the release of cytolytic granules containing granzyme and perforin; the second mechanism relates to cytolysis as a consequence of target cell expression of cell surface receptors for FasL or Trail. The data reported herein suggest that this second mechanism (FasL and/or Trail involvement) may mediate the enhanced effectiveness of effector cells that are adapted to TME conditions.

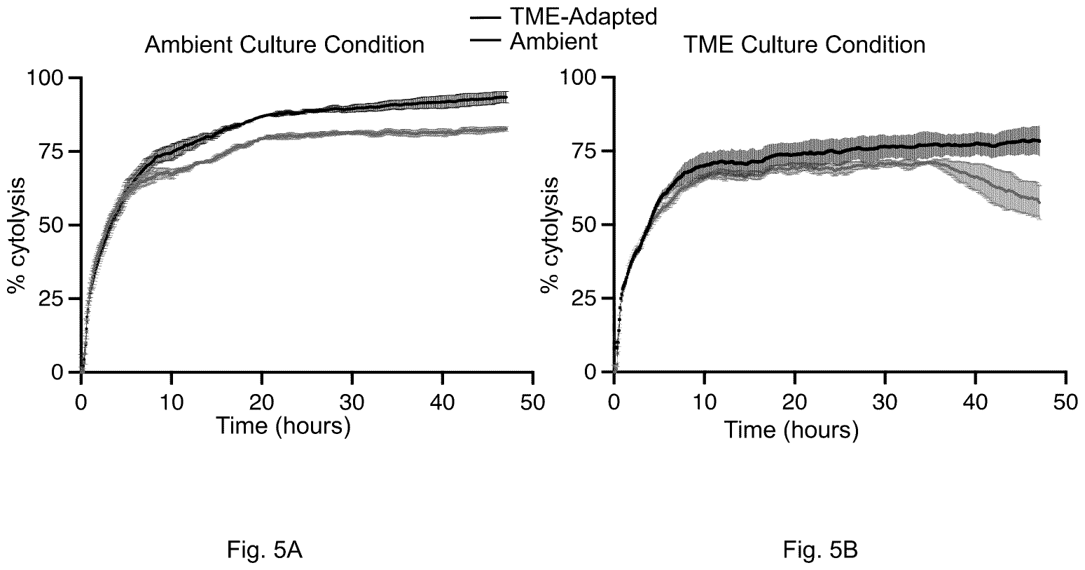

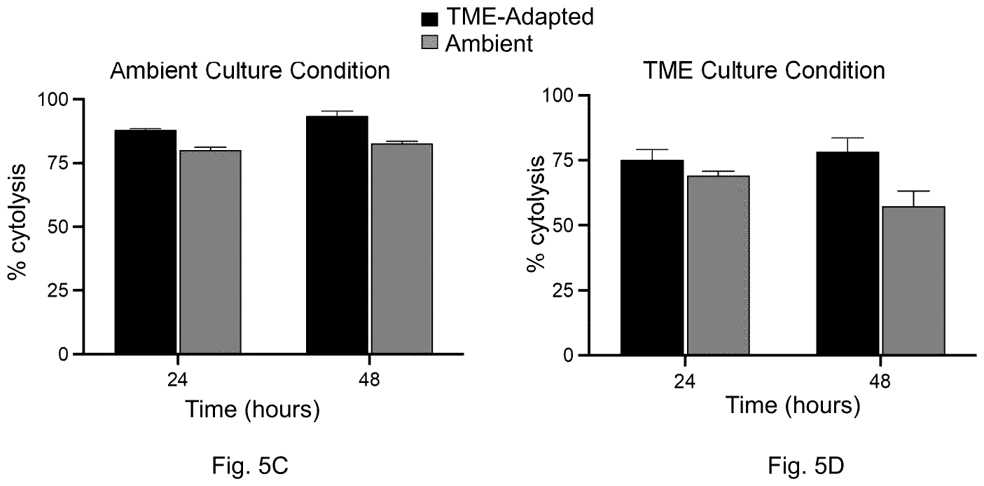

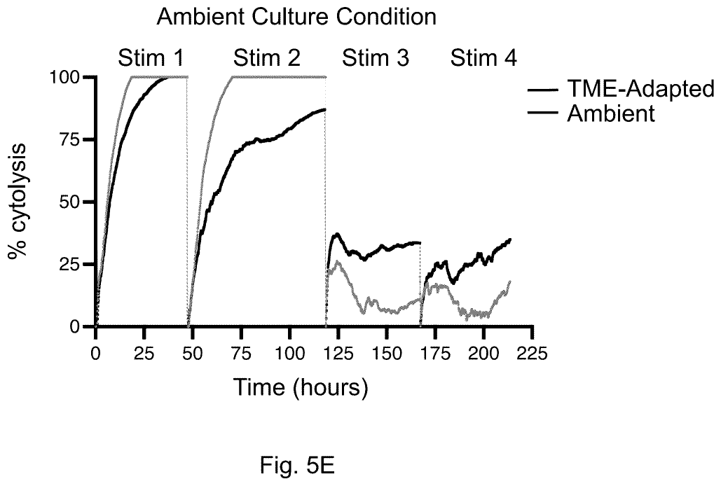

FIGS. 5A-5B show target cells being exposed to an effector cell population under both ambient (FIG. 5A) and TME (FIG. 5B) conditions. The effector cells were either CAR-T cells grown under ambient conditions or cells that have been adapted to TME conditions of ambient O2 and 4 PSIG. These effector cell conditions are identified in Table 3, below. The effector cells were added to target cells at a ratio of 10:1 (effector cells to target cells), and subsequent cytolysis of target cells was measured over time. ROR1-CAR-T cells grown under TME conditions exhibited increased cytotoxic activity. When experiments were repeated under TME conditions, the differences between ROR1-CAR-T cell culture conditions were only observable at later time points.

FIGS. 5C-5D show these cytolysis data quantified at two times points. This analysis shows that ROR1-CAR-T cells grown under TME conditions exhibit increased cytolysis at 24 and 48 hours after initiation of coculture with SKOV3 cells, as compared to ROR1-CAR-T cells grown under ambient conditions. This increased cytolysis is observed when coculture occurs at either ambient or TME conditions, with the difference between ROR1-CAR-T cell culture conditions being more evident at the later time point in TME conditions.

FIG. 5E shows effector cell populations being repeatedly exposed to target cells under both ambient conditions, for a total of four stimulations. The effector cells were either CAR-T cells grown under ambient conditions or cells that have been adapted to TME conditions of 3% O2 and 4 PSIG. The effector cells were added to target cells at a ratio of 10:1 (effector cells to target cells), and subsequent cytolysis of target cells was measured over time. Though CAR-T cells expanded under TME conditions exhibited reduced cytolysis at stimulation one and two, as compared to CAR-T cells expanded under ambient conditions, the TME-adapted CAR-T cells exhibited enhanced cytolytic function in the last two stimulations.

Compared to the NK-92 cell cultured in a conventional CO2 incubator or long-term adapted NK-92 cells under tumor microenvironment (TME conditions: 1% O2 and 2 PSIG) (TME adapted NK-92) showed increased cell cytotoxicity and potency in killing populations of non-small lung cancer cell line A549.

TABLE 1
Experimental Design: NK-92 Effector Cell Environment
Prior to Co-Culturing with A459 Target Cells

| Group No. and | Conditions of effector cell (NK-92) environment | |
|---|---|---|
| Chart Label | prior to contacting target A459 cells | |
| 1. Parental | CO2 incubator | Normal CO2 incubator conditions |
| | Ambient O2 and | for effector cells before contacting |
| | Ambient Pressure | target cells. |
| 2. Adapted | Avatar Incubator | Long term adapted: preconditioned |
| | 1% O2 & 2 PSIG | for 3 months (~70 generations) in |
| | | 1% O2 and 2 PSIG before |
| | | contacting target cells. |

Cytolytic effectiveness of NK-92 cell populations (effector cells) against a non-small lung cancer cell population (A549) cells was tested by co-culturing them. NK-92 or NK-92-derivative effector cells are added to target cells in a 96 well plate 200 uL volume assay. The already-present target A549 cell density is consistently 10K cells/well; the number of attacking effector cells varies from 10K to 100K cells/well.

TABLE 2
Experimental Design: a focus on the ratio
of effector cells to target cells

| Ratio of | | |
|---|---|---|
| Effector Cells to | Effector Cells | Target Cells |
| Target Cells | added/well | present/well |
| 0:1 | — | 10K |
| 1:1 | 10K | 10K |
| 2:1 | 20K | 10K |
| 5:1 | 50K | 10K |
| 10:1 | 100K | 10K |

### Cytolytic Assay Method

Target cancer cell lines (e.g., A549) were adapted to tumor microenvironment conditions in the AVATAR system and labeled with a proliferation dye and plated in 96 well E-plates in duplicate or triplicate. Optimal initial cell densities were previously determined in optimization experiments. The target cells are then incubated overnight in standard or TME conditions to allow time for adherence to the E-plates.

At 24 hrs, NK-92 cells (TME-Adapted or standard incubation) are inoculated into the wells already containing target cells at various effector to target ratios. Variables may include the presence or absence of stimulatory cytokines or drug (immunotherapy or small molecule inhibitors). The co-culture is then incubated at standard or TME conditions and impedance readings recorded over the course of several days at various timepoints (4 hrs, 24 hrs, etc.). Supernatants were harvested for cytokine analysis and the cells are collected for functional analysis.

Functional analytical assays include: CellTiter Glo for overall proliferation of the co-culture or flow cytometry to observe the specific lysis of the target cells and phenotypic analysis of the NK-92 cells.

As described above, impedance is measured by reactance, and used as a surrogate for cell density. Reactance data can also be transformed into a surrogate for cytolysis. The difference in reactance between an experimental group that is lower than an appropriate control group can be reasonably understood as a measure of cells missing by way of cytolysis. This same “missing cells” approach can be applied to flow cytometry analysis, in addition to other characteristics of a cell population, such as vital staining or surface marker staining.

By way example, a step-by-step a cytolytic potency assay method is outlined below.
1. Plated target cancer cell lines are labeled with a proliferation dye, then incubated overnight in standard or TME conditions. Incubation overnight allows the cells to attach to wells in multi-well cell culture plates (E-Plates, by Agilent) that include an electronic grid within the bottom surface of each well. Cell density for plating is determined by preliminary/optimization experiments.
2. At 24 hrs, effector cells (NK-92-derivative cells and appropriate controls) cells are added at various effector-to-target ratios, i.e., 10:1, 5:1, 2:1 etc. as determined by preliminary/optimization experiments.
3. Stimulation by way of bioactive agents may be optionally added at this point.
4. Multiples of each plate may be set up to do functional assays (one set per timepoint), including CellTiter Glo for proliferation or flow cytometry to determine specific lysis of the target cells and functional activity of the NK-92s by phenotype, and cytokine analysis of the cell culture supernatants.
5. Impedance readings are taken over the course of 4 hrs to 5 days in standard or TME conditions. Assay timepoints may include: 4 hrs, 24 hrs, 48 hrs, etc.
6. At each timepoint, the cells are removed and analyzed as described in step 4.

### CAR-T Cell Populations: Experimental Details

Compared to the ROR1-CAR-T cells cultured in a conventional CO2 incubator, ROR1-CAR-T cells cultured under modified oxygen and/or pressure conditions (TME adapted CAR-T cells) showed increased cell cytotoxicity and potency in killing populations of ovarian cancer cell line SKOV3. These experiments were performed using a similar approach to the Cytolytic Assay described above.

TABLE 3
Experimental Design: Focus on ROR1-CAR-T Effector Cell Environment
Prior to Co-Culturing with SKOV3 Target Cells

| Group No. and | Conditions of effector cell (CAR-T cells) environment | |
|---|---|---|
| Chart Label | prior to contacting target SKOV3 cells | |
| 1. Standard | CO2 incubator | Normal CO2 incubator conditions |
| | Ambient O2 and | for effector cells before contacting |
| | Ambient Pressure | target cells. |
| 2. Adapted | Avatar Incubator | Conditioned for 9 days in |
| | Ambient O2 & | Ambient O2 & 4PSIG or 3% O2 & |
| | 4PSIG or 3% O2 | 4PSIG before contacting target cells. |
| | & 4PSIG | |

Any one or more features or steps of any embodiment of the inventions disclosed herein can be combined with any one or more other features of any other described embodiment of the invention without departing from the scope of the invention. It should also be understood that the inventions are not limited to the embodiments that are described or depicted herein for purposes of exemplification, but are to be defined only by a fair reading of claims appended to the patent application, including the full range of equivalency to which each element thereof is entitled. Some theoretical considerations of the inventors have been advanced in this application; these theoretical considerations are offered strictly for the purpose of conveying concepts underlying the inventions, not as support for the claims, all of which stand wholly independent of any theoretical considerations.

A first embodiment of the invention includes a method of developing a derivative cell population from a parent cell population, wherein the derivative cell population expresses a phenotype adapted to a hypoxic and hyperbaric environment. Embodiments of the method include culturing a population of the parent cell population under the hypoxic and a hyperbaric condition in vitro for a sufficient time to permit a phenotypically adapted derivative cell population to emerge. A hypoxic and a hyperbaric condition may be referred to as a tumor microenvironment (TME) condition. In some embodiments, a phenotypically adapted cell population may include cells that have been epigenetically reprogrammed. “Sufficient time” for a derivative population to emerge refers to time in serial cell culture, during which time a sufficient number of cell generations have transpired.

In some embodiments, a “cell population” may be highly homogeneous, and understood to be a “cell line” that shows a high degree of phenotypic stability. In some embodiments, the phenotypically adapted derivative cell population comprises an epigenetic alteration that underlies an expression of phenotypic features that distinguish the derivative cell population from the parent cell population. In some embodiments, the phenotypically adapted derivative cell population comprises a genomic alteration that underlies an expression of phenotypic features that distinguish the derivative cell population from the parent cell population.

The phenotypically adapted derivative cell population expresses phenotypic features that distinguish it from the parent cell population. The phenotypic features of the derivative cell line (as particularly expressed under the hypoxic and hyperbaric condition) that distinguish it from the phenotypic features of the parent cell population (i.e., a population without a history of in vitro exposure to the hypoxic and a hyperbaric condition) include one or more of (a) an increased growth rate, (b) an increased viability, (c) an altered metabolic activity profile, or (d) an altered differentiated functionality particular to the parent cell population. By way of example, an altered functionality of an immune cell population may include any cellular behavior that enhances or improves immunologic effectiveness of the derivative cells against target cells, either in vitro or in vivo, as within a solid tumor of a cancer patient being treated with the derivative cell population.

For clarity, noting that a derivative cell population considered to have promise for a human therapeutic application need not show all of the enumerated (a-d) features. And it is also a possibility that the advantage of (d) an elevated immunologic function could outweigh features that may not necessarily seen as advantageous, such as a lower growth rate.

In some of these embodiments, the parent cell population includes an effector immune cell population. In some of these embodiments, the immune cell population includes a natural killer T cell population or a derivative thereof. And in some of these embodiments, the effector immune cell population is derived from a human patient. In some of the embodiments, the effector cell is an immune cell population that specifically targets patient-specific cancer cells residing in a solid tumor.

### Hypoxia Response Elements and Hypoxia-Induced Factors

In some embodiments of the method, the distinguishing phenotypic features of the derivative cell population that permit the phenotypically adapted derivative cell population to emerge include any one or more of (a) transcriptional control, (b) translational control, or (c) mRNA or protein degradation rate.

The distinguishing phenotypic features of the derivative cell population may include an elevated level of a hypoxia response element activation within a hypoxia-induced factor (HIF) gene. Distinguishing phenotypic features of the derivative cell population may include an elevated expression of a hypoxia-induced factor. By way of example, a hypoxia-induced factor may include any one or more of HIF-1a, HIF-1b, HIF-2a, HIF-2b, HIF-1a, or HIF-1b.

In other embodiments, the expression of phenotypic features in the derivative cell population may be mediated by factors other than the HIF factors, such as a member of the Piezo protein family (mechanosensitive ion channel components). In another example, solute carrier (SLC) proteins that act as anion exchange based membrane transporters may become factors that mediate hypoxia effects or responses.

### Immunologic Functionality

In some embodiments of the method, the parent cell population includes an immune cell population having an immune-system functionality, and wherein, in comparison to the immune functionality of the parent cell population, the derivative cell population includes an elevated level of the immune functionality against a target cell population. By way of a clinical application as a related method, when infused into a patient with a solid tumor that includes the target cell population, the elevated level of immune functionality of the derivative cell population may deliver an effective therapeutic attack against the target cell population.

### Cytolytic Activity

In some embodiments of the method, the altered level of immunologic functionality of the derivative cell population may include any one or more of an increased cytolytic capability against a target cell population or a target cell apoptotic trigger. By way of a clinical application as a related method, the target cell population may include a resident cancer cell population within a primary solid tumor or in the form of a dispersed population of cells that have metastasized from the primary tumor.

### Immunosuppressive Activity

In alternative embodiments of the method, the altered level of immunologic functionality of the derivative cell population includes an increased level of an immunosuppressive effect on a target cell population.

### Expression of the Derivative Cell Population in Various Environments

In some embodiments of the method, the distinguishing phenotypic features of the derivative cell population, in comparison to the parent cell population, are expressed under hypoxic and hyperbaric conditions in an in vitro environment. In some embodiments of the method, the distinguishing phenotypic features of the derivative cell population, in comparison to the parent cell population, are expressed under hypoxic and hyperbaric conditions in an in vivo environment. In some embodiments of the method, the in vivo environment includes a tumor site of a patient into which the derivative cell population has been therapeutically infused.

### Parent Immune Effector Model Cell Populations

In some embodiments of the method, the parent cell population includes a lymphoid cell developmental lineage. In embodiments, the parent cell population includes a T cell developmental lineage. In embodiments, the parent cell population includes a natural killer (NK) cell population or a derivative thereof. In some embodiments, the parent cell population is a human-derived cell population.

Model Cell Populations with and without Transfection

In some embodiments of the method, allowing emergence of a hyperbaric and hypoxic condition-adapted derivative cell population occurs in the absence of a concurrent transfective procedure during the cell culture duration. In other embodiments, the modulation of the phenotype of the parent population to the phenotype of the parent-derivative population occurs in conjunction with a concurrent transfective procedure during the cell culture duration. In still other embodiments, the parent cell population is transfected prior to exposure to culturing under hypoxic and/or hyperbaric conditions.

### Cell Banking, Cell Bank Testing

In some embodiments of the method, after the phenotypically adapted cell derivative population has emerged from the parent population, the method further includes cryogenically preserving a quantity of the derivative cell population as a cell bank in cryogenic vials.

In some embodiments of the method, the method further includes thawing one or more of the cryogenic vials, culturing the thawed cells, and testing the thawed cells to determine whether the hypoxia and hyperbaric adaptations are substantially identical to the adaptations of the cell population prior to cryogenic preservation.

### Further Embodiments

A second embodiment of the invention includes a method of developing a derivative call population from a parent cell population, wherein the derivative cell population expresses a phenotype adapted to a hypoxic environment. This method embodiment includes culturing a population of the parent cell population under a hypoxic condition in vitro for a sufficient number of generations or time to permit a phenotypically adapted derivative cell population to emerge during a cell culture duration.

The phenotypically adapted derivative cell population expresses phenotypic features that distinguish it from the parent cell population. The phenotypic features of the derivative cell population, as expressed under the hypoxic and hyperbaric condition, that distinguish it from the phenotypic features of the parent cell population (without a history of exposure to the hypoxic and a hyperbaric condition) include one or more of (a) an increased growth rate, (b) an increased viability, (c) an altered metabolic activity profile, or (d) an altered differentiated functionality particular to the parent cell population.

For clarity, this second embodiment of the method is directed to hypoxic conditions in vitro, without reference to presence or absence of hyperbaric conditions. All features enumerated in the context of the first method embodiment may be included in the scope of this second embodiment.

A third embodiment of the invention includes a method of developing a derivative call population from a parent cell population, wherein the derivative cell population expresses a phenotype adapted to a hyperbaric environment. This method embodiment includes culturing a population of the parent cell population under a hyperbaric condition in vitro for a sufficient number of generations or time to permit a phenotypically adapted derivative cell population to emerge during a cell culture duration,

The phenotypically adapted derivative cell population expresses phenotypic features that distinguish it from the parent cell population. The phenotypic features of the derivative cell population, as expressed under the hypoxic and hyperbaric condition, that distinguish it from the phenotypic features of the parent cell population (without a history of exposure to the hypoxic and a hyperbaric condition) include one or more of (a) an increased growth rate, (b) an increased viability, (c) an altered metabolic activity profile, or (d) an altered differentiated functionality particular to the parent cell population.

For clarity, this third embodiment of the method is directed to hyperbaric conditions in vitro, without reference to presence or absence of hypoxic conditions. All features enumerated in the context of the first method embodiment may be included in the scope of this second embodiment.

A fourth embodiment of the invention includes a method of determining a level of cytolytic effectiveness of an effector cell population against a target cell population. This embodiment of the method includes: culturing the target cell population in vitro; and culturing the effector cell population in vitro, wherein the effector cell population has been phenotypically adapted with respect to a parent cell population from which it was derived, wherein the adapted phenotype includes an adaptation to a hyperbaric and hypoxic environment. The method embodiment continues by combining a portion of the cultured target cell population and a portion of the cultured effector cell population together to form a combined culture; culturing the combined target cell population and the effector cell population together for a culture duration; and determining the level of cytolysis of the target cell population as a consequence of the presence of the effector cell population.

In some embodiments of the fourth embodiment, the effector cell population includes natural killer T cell or a derivative thereof. In some embodiments, the target cell population includes a cancer cell population. In some embodiments, the cancer cell population is sourced from a human cancer patient. In some embodiments, the target cell population includes a population of cancer cells resident within a solid tumor.

Some embodiments of the method are suitable as a companion diagnostic (CDx) assay that measures a level of cytolytic effectiveness that correlates with a level of therapeutic effectiveness of the effector cell population against a human cancer cell population within a tumor, within a patient.

A fifth embodiment of the invention includes a method of treating cancer in a patient includes providing an effector cell population that has, in response to being cultured in a hyperbaric and hypoxic condition, has undergone a phenotypic adaptation that supports one or more expressed features that, in comparison to a cell population from which the effector cell population was derived, improve a performance of the effector cell population under hypoxic and/or hyperbaric conditions, or under tumor microenvironment conditions, wherein the performance of the effector cell population includes an immunologic functionality against a target cell population. The method embodiment may further include infusing a physiologically effective amount of the effector cell population into a patient to treat a tumor includes the target cell.

A sixth embodiment of the invention includes a cell population as a product that is adapted to a hypoxic and hyperbaric condition. Some embodiments are in the form of an immune cell product. Criteria that apply to this product follow.

The adapted cell population is derived from an originating parent cell population not-adapted to a hypoxic and/or a hyperbaric condition; the adapted cell population, in transitioning from the originating parent cell population, has been cultured for a sufficient number of generations under a hypoxic and a hyperbaric condition to permit a phenotypic adaptation to occur that underlies adaptation to the hypoxic and a hyperbaric condition. In one embodiment, a sufficient number of generations may include 5, 10, 15, or 20 generations.

The phenotypically adapted derivative cell population has phenotypic features that distinguish it from the parent cell population, and the phenotypic features of the derivative cell population, as expressed under the hypoxic and hyperbaric condition, that distinguish it from the phenotypic features of the parent cell population (without a history of exposure to the hypoxic and a hyperbaric condition) includes one or more of (a) an increased growth rate, (b) an increased viability, (c) an altered metabolic activity profile, or (d) an altered differentiated functionality particular to the parent cell population.

A seventh embodiment of the invention includes a method cell banking that includes freezing a cell population of interest in a liquid cryopreserving medium to form a frozen cell-banked cell population; thawing the frozen cell population into a liquid cell culture medium; culturing the thawed cell population in a cell culture system that can regulate (a) a total gas atmospheric pressure and (b) a hypoxic gas composition to form a hyperbaric and hypoxic gas environment that surrounds a cell culture container in the system, wherein the total gas atmospheric pressure and the hypoxic gas composition are regulated independently of each other. The method may further include expanding the cell population within the cell culture system, and within the hyperbaric and hypoxic gas environment, to yield a cell population expanded to a desired level.

In embodiments, prior to freezing the cell population of interest, the method includes culturing the population in the hyperbaric and hypoxic gas environment provided by the cell culture system.

In other embodiments, the cell population of interest is a population derived from a parent population, the cell population of interest being phenotypically distinct from the parent population. Distinctness from the parent population may include d any one or more of (a) an increased growth rate, (b) an increased viability, (c) an altered metabolic activity profile, (d) an altered morphology, or (e) an altered functional feature with respect to that of the parent cell population.

An eighth embodiment of the invention includes a pharmaceutical composition that includes a cell population suitable for infusion into a patient, the cell population having an adaptation to a hyperbaric and hypoxic condition wherein the adapted cell population is derived from an originating parent cell population not-adapted to a hypoxic and/or a hyperbaric condition. With reference to the cell population included within the pharmaceutical composition, the follow criteria apply.

The adapted cell population, in transitioning from the originating parent cell population, has been cultured for a sufficient number of generations under a hypoxic and a hyperbaric condition to permit a phenotypic adaptation to occur that underlies adaptation to the hypoxic and a hyperbaric condition; the phenotypically adapted derivative cell population includes phenotypic features that distinguish it from the parent cell population. Further, the phenotypic features of the derivative cell population, as expressed under the hypoxic and hyperbaric condition, that distinguish it from the phenotypic features of the parent cell population (without a history of exposure to the hypoxic and a hyperbaric condition) includes one or more of (a) an increased growth rate, (b) an increased viability, (c) an altered metabolic activity profile, or (d) an altered differentiated functionality particular to the parent cell population.

A ninth embodiment of the invention includes a modified immune cell population derived from a native immune cell population, modified for increased cytolytic activity towards a cancer cell population relative to the native immune cell population, the modified immune cell population comprising: a decreased expression of a first protein, wherein the first protein comprises HIF2α, granzyme B (GZMB), perforin, FasL, or GNYL; and an increased expression of a second protein, wherein the protein comprises HIF1α, TRAIL, FasL, GNLY, or GZMM.

In some embodiments, the modified immune cell population does not differ from the native immune cell population with respect to expression of beta-actin. In some embodiments, a decreased expression of granzyme B, wherein the decreased expression of granzyme B comprises a 5, 10, 15, 20, 25, 30, 35, 40, 45, 50, 55, 60, 65, 70, 75, 80, 85, 90, or 95% of a native level of granzyme B expression. In some embodiments, a decreased expression of perform, wherein the decreased expression of perform comprises a 5, 10, 15, 20, 25, 30, 35, 40, 45, 50, 55, 60, 65, 70, 75, 80, 85, 90, or 95% of a native level of perforin expression. In some embodiments, an increased expression of TRAIL, wherein the increased expression of TRAIL comprises a 5, 10, 15, 20, 25, 30, 35, 40, 45, 50, 55, 60, 65, 70, or 75% increase in a native level TRAIL expression. In some embodiments, an increased expression of HIF1α, wherein the increased expression of HIF1α comprises a 200, 250, 300, 350, 400, 450, 500, 600, 700, 800, 900, or 1000% increase in a native level HIF1α expression. In some embodiments, a decreased expression of HIF2α, wherein the decreased expression of HIF2α comprises a 10, 20, 30, 40, 50, 60, 70, 80, 90, or 100% of a native level of HIF2α expression. In some embodiments, a decreased expression of FASL, wherein the decrease in expression of FASL comprises 5, 10, 15, 20, 25, 30, 35, 40, 45, 50, 55, 60, 65, 70, 75, 80, 85, 90, or 95% of a native level of FASL expression. In some embodiments, an increased expression of FasL, wherein the increased expression of FasL comprises a 10, 20, 30, 40, 50, 60, 70, 80, 90, or 100, 110, 120, 125, 130, 140, 150, 160, 170, 180, 190, or 200% increase in a native level FasL expression. In some embodiments, a decreased expression of GNLY, wherein the decrease in expression of GNLY comprises 5, 10, 15, 20, 25, 30, 35, 40, 45, 50, 55, 60, 65, 70, 75, 80, 85, 90, or 95% of a native level of GNLY expression. In some embodiments, an increased expression of GNLY, wherein the increased expression of GNLY comprises 50, 60, 70, 80, 90, or 100, 110, 120, 125, 130, 140, 150, 160, 170, 180, 190, 200, 250, 300, 350, 400, 450, or 500% increase in a native level GNLY expression. In some embodiments, an increased expression of GZMM, wherein the increased expression of GZMM comprises 5, 10, 15, 20, 25, 30, 35, 40, 45, 50, 60, 70, 80, 90, or 100, 110, 120, 125, 130, 140, 150, 160, 170, 180, 190, 200, 210, 220, 230, 240, 250, 260, 270, 280, 290, 300, 325, 350, 375, or 400% increase in a native level GZMM expression.

In some embodiments, the modified immune cell population comprises T cells, or wherein the native immune cell population comprises T cells. In some embodiments, the modified immune cell population comprises natural killer T cells, or wherein the native immune cell population comprises natural killer T cells. In some embodiments, the modified immune cell population comprises NK-92 cells, or wherein the native immune cell population comprises NK-92 cells. In some embodiments, an increased expression of HIF1α, TRAIL, FasL, GNLY, and GZMM. The modified immune cell population of the immediately preceding claim further comprising a decreased expression of HIF2α, GZMB, and Perform. The modified immune cell population of the immediately preceding claim further comprising an increased expression of HIF1α, TRAIL, FasL, GNLY, and GZMM in the approximate amounts of 610% HIF1α, 125% TRAIL, 150% FasL, 300% GNLY, and 300% GZMM of native expression levels. The modified immune cell population of the immediately preceding claim further comprising a decreased expression of HIF2α, GZMB, and Perforin in the approximate amounts of 90% HIF2α, 25% GZMB, and 35% Perform of a native level of expression of native expression levels. In some embodiments, the modified immune cell population is long term adapted to hypoxic conditions. In some embodiments, an increased expression of HIF1α, TRAIL, and GZMM. The modified immune cell population of the immediately preceding claim further comprising a decreased expression of FasL, Perform, and GNYL. The modified immune cell population of the immediately preceding claim an increased expression of HIF1α, TRAIL, and GZMM in the approximate amounts of 290% HIF1α, 150% TRAIL, and 175% GZMM of native expression levels. The modified immune cell population of the immediately preceding claim further comprising a decreased expression of FasL, Perforin, and GNYL in the approximate amounts of, 33% FasL, 50% Perforin, and 80% GNYL of native expression levels. In some embodiments, the modified immune cell population is short term adapted to hypoxic conditions. In some embodiments, an increased expression of HIF1α, TRAIL, FasL, GNLY, and GZMM. The modified immune cell population of the immediately preceding claim further comprising a decreased expression of HIF2α, GZMB, FasL, and Perform. The modified immune cell population of the immediately preceding claim an increased expression of HIF1α, TRAIL, FasL, GNLY, and GZMM in the approximate amounts of increased expression of 925% HIF1α, 150% TRAIL, 135% FasL, 500% GNLY, and 225% GZMM of native expression levels. The modified immune cell population of the immediately preceding claim further comprising a decreased expression of FasL, Perform, GNYL and GZMM in the approximate amounts of 80% HIF2α, 30% Perforin, 500% GNYL and 220% GZMM of native expression levels. In some embodiments, the modified immune cell population is long term adapted to hypoxic and hyperbaric conditions. In some embodiments, an increased expression of HIF1α, TRAIL, and GZMM. The modified immune cell population of the immediately preceding claim further comprising a decreased expression of FasL, GNLY, and Perform. The modified immune cell population of the immediately preceding claim an increased expression of HIF1α, TRAIL, and GZMM in the approximate amounts of increased expression of 525% HIF1α, 125% TRAIL, and 175% GZMM of native expression levels. The modified immune cell population of the immediately preceding claim further comprising a decreased expression of FasL, GNLY, and Perforin in the approximate amounts of 25% FasL, 50% GNLY, and 33% Perforin of native expression levels. In some embodiments, the modified immune cell population is short term adapted to hypoxic and hyperbaric conditions. In some embodiments, the modified immune cell population induces cytolysis of cancer cells by death receptor mediated cytotoxicity. In some embodiments, death receptor mediated cytotoxicity is induced by increased expression of FasL or TRAI1. In some embodiments, the modified immune cell population exhibits increased cytolytic activity towards cancer cells at ratios 1:1; 2:1; 5:1; and 10:1 of modified immune cells to cancer cells. In some embodiments, the increased cytolytic activity towards cancer cells comprises inducing cytolysis of 5, 10, 15, 20, 25, 30, 35, 40, 45, 50, 55, 60, 65, 70, 75, 80, 85, 90, 95, 99, or 100% of cancer cells in solid tumor.

### Numbered Embodiments

1. A method of developing a derivative cell population from a parent cell population, wherein the derivative cell population expresses a phenotype adapted to a hypoxic and hyperbaric environment, the method comprising:
   culturing a population of the parent cell population for at least 10 generations under the hypoxic and a hyperbaric condition in vitro for a sufficient time to permit a phenotypically-adapted derivative cell population to emerge,
      wherein the phenotypically-adapted derivative cell population comprises phenotypic features that distinguish it from the parent cell population, and
      wherein the phenotypic features of the derivative cell population, as expressed under the hypoxic and hyperbaric condition, that distinguish it from the phenotypic features of the parent cell population (without a history of exposure to the hypoxic and a hyperbaric condition) comprise one or more of (a) an increased growth rate, (b) an increased viability, increased cytolytic activity (c) an altered metabolic activity profile, or (d) an altered differentiated functionality that is particular to the parent cell population.
2. The method of embodiment 1 wherein the sufficient time to permit a phenotypically-adapted derivative cell population to emerge also includes a sufficient number of cell generations for the phenotypically-adapted derivative cell population to emerge.
3. The method of embodiment 1 the phenotypically-adapted derivative cell population comprises an epigenetic alteration that underlies an expression of phenotypic features that distinguish the derivative cell population from the parent cell population.
4. The method of embodiment 1 the phenotypically-adapted derivative cell population comprises a genomic alteration that underlies an expression of phenotypic features that distinguish the derivative cell population from the parent cell population.
5. The method of embodiment 1 wherein the parent cell population comprises an effector immune cell population.
6. The method of embodiment 5 wherein the effector immune cell population comprises a natural killer T cell population or a derivative thereof.
7. The method of embodiment 1 wherein the effector immune cell population is derived from a human patient.
8. The method of embodiment 1 wherein the distinguishing phenotypic features of the derivative cell population that permit the phenotypically-adapted derivative cell population to emerge comprise any one or more of (a) transcriptional control, (b) translational control, or (c) mRNA or protein degradation rate.
9. The method of embodiment 1 wherein the distinguishing phenotypic features of the derivative cell population comprise an elevated level of a hypoxia response element activation within a hypoxia-induced factor (HIF) gene.
10. The method of embodiment 1 wherein the distinguishing phenotypic features of the derivative cell population comprise an elevated expression of a hypoxia-induced factor.
11. The method of embodiment 10 wherein the hypoxia-induced factor comprises any one or more of HIF-1a, HIF-1b, HIF-2a, HIF-2b, HIF-1a, or HIF-1b.
12. The method of embodiment 1 wherein the parent cell population comprises an immune cell population having an immune-system functionality, and wherein, in comparison to the immune functionality of the parent cell population, the derivative cell population comprises an elevated level of the immune functionality against a target cell population.
13. The method of embodiment 12 wherein, when infused into a patient with a solid tumor comprising the target cell population, the elevated level of immune functionality delivers an effective therapeutic attack against the target cell population.
14. The method of embodiment 12 wherein the altered level of immunologic functionality of the derivative cell population comprises any one or more an increased cytolytic capability against a target cell population or an apoptotic trigger.
15. The method of embodiment 14 wherein the target cell population comprises a cancer cell population within a primary solid tumor or within a population of cells that have metastasized from the primary tumor.
16. The method of embodiment 1 wherein the altered level of immunologic functionality of the derivative cell population comprises an increased level of an immunosuppressive effect on a target cell population.
17. The method of embodiment 1 wherein the distinguishing phenotypic features of the derivative cell population, in comparison to the parent cell population, are expressed under hypoxic and hyperbaric conditions in an in vitro environment.
18. The method of embodiment 1 wherein the distinguishing phenotypic features of the derivative cell population, in comparison to the parent cell population, is expressed under hypoxic and hyperbaric conditions in an in vivo environment.
19. The method of embodiment 18 wherein the in vivo environment comprises a tumor site of a patient into which the derivative cell population has been therapeutically infused.
20. The method of embodiment 1 wherein the parent cell population comprises a lymphoid cell developmental lineage.
21. The method of embodiment 20 wherein the parent cell population comprises a T cell developmental lineage.
22. The method of embodiment 21 wherein the parent cell population comprises a natural killer (NK) cell population or a derivative thereof.
23. The method of embodiment 1 wherein the parent cell population is a human-derived cell population.
24. The method of embodiment 1 wherein allowing emergence of a hyperbaric and hypoxic condition-adapted derivative cell population occurs in the absence of a concurrent transfective procedure during the cell culture duration.
25. The method of embodiment 1 wherein the modulation of the phenotype of the parent population to the phenotype of the parent-derivative population occurs in conjunction with a concurrent transfective procedure during the cell culture duration.
26. The method of embodiment 1 wherein the parent cell population was transfected prior to exposure to culturing under hypoxic and/or hyperbaric conditions.
27. The method of embodiment 1, wherein after the phenotypically-adapted derivative cell population has emerged from the parent population, the method further comprises cryogenically preserving a quantity of the cell population as a cell bank in one or more cryogenic vials.
28. The method of embodiment 27, wherein the method further comprises thawing one or more of the cryogenic vials, culturing the thawed cells, and testing the thawed cells to determine whether the hypoxia and hyperbaric adaptations are substantially identical to the adaptations of the cell population prior to cryogenic preservation.
29. A method of developing a derivative call population from a parent cell population, wherein the derivative cell population expresses a phenotype adapted to a hypoxic environment, the method comprising:
   culturing a population of the parent cell population under a hypoxic condition in vitro for a sufficient number of generations to permit a phenotypically-adapted derivative cell population to emerge during a cell culture duration,
      wherein the phenotypically-adapted derivative cell population comprises phenotypic features that distinguish it from the parent cell population, and
      wherein the phenotypic features of the derivative cell population, as expressed under the hypoxic condition, that distinguish it from the phenotypic features of the parent cell population (without a history of exposure to the hypoxic condition) comprise one or more of (a) an increased growth rate, (b) an increased viability, (c) an altered metabolic activity profile, or (d) an altered differentiated functionality that is particular to the parent cell population.
30. A method of developing a derivative call population from a parent cell population, wherein the derivative cell population expresses a phenotype adapted to a hyperbaric environment, the method comprising:
culturing a population of the parent cell population under a hyperbaric condition in vitro for a sufficient number of generations to permit a phenotypically-adapted derivative cell population to emerge during a cell culture duration,
   wherein the phenotypically-adapted derivative cell population comprises phenotypic features that distinguish it from the parent cell population, and
   wherein the phenotypic features of the derivative cell population, as expressed under the hyperbaric condition, that distinguish it from the phenotypic features of the parent cell population (without a history of exposure to the hyperbaric condition) comprise one or more of (a) an increased growth rate, (b) an increased viability, (c) an altered metabolic activity profile, or (d) an altered differentiated functionality that is particular to the parent cell population.
31. A method of determining a level of cytolytic effectiveness of an effector cell population against a target cell population, the method comprising:
   culturing the target cell population in vitro;
   culturing the effector cell population in vitro, wherein the effector cell population has been phenotypically-adapted with respect to a parent cell population from which it was derived, wherein the phenotypically-adapted phenotype comprises an adaptation to a hyperbaric and hypoxic environment;
   combining a portion of the cultured target cell population and a portion of the cultured effector cell population together to form a combined culture;
   culturing the combined target cell population and the effector cell population together for a culture duration; and
   determining the level of cytolysis of the target cell population as a consequence of the presence of the effector cell population.
32. The method of embodiment 31 wherein the effector cell population comprises natural killer T cell or a derivative thereof.
33. The method of embodiment 31 wherein the target cell population comprises a cancer cell population.
34. The method of embodiment 34 wherein the cancer cell population is sourced from a human cancer patient.
35. The method of embodiment 31 wherein the target cell population comprises a population of cancer cells within a solid tumor.
36. The method of embodiment 31 wherein the method comprises an assay, suitable as a companion diagnostic (CDx) assay, measures a level of cytolytic effectiveness that corresponds to a level of therapeutic effectiveness of the effector cell population against a human cancer cell population within a tumor, within a patient.
37. A method of treating cancer in a patient comprising:
   providing an effector cell population that has, in response to being cultured in a hyperbaric and hypoxic condition, has undergone a phenotypic adaptation to TME condition that supports one or more adaptations that, in comparison to a cell population from which the effector cell population was derived, improve a performance of the effector cell population under hypoxic and/or hyperbaric conditions, wherein the performance of the effector cell population comprises an immunologic functionality against a target cell population; and
   infusing a physiologically effective amount of the effector cell population into a patient to treat a tumor comprising the target cell.
38. A cell population adapted to a hypoxic and hyperbaric condition:
   wherein the adapted cell population is derived from an originating parent cell population not-adapted to a hypoxic and/or a hyperbaric condition,
   wherein the adapted cell population, in transitioning from the originating parent cell population, has been cultured for a sufficient number of generations under a hypoxic and a hyperbaric condition to permit a phenotypic adaptation to occur that underlies adaptation to the hypoxic and a hyperbaric condition,
   wherein the phenotypically TME-adapted derivative cell population comprises phenotypic features that distinguish it from the parent cell population, and
      wherein the phenotypic features of the derivative cell population, as expressed under the hypoxic and hyperbaric condition, that distinguish it from the phenotypic features of the parent cell population (without a history of exposure to the hypoxic and a hyperbaric condition) comprise one or more of (a) an increased growth rate, (b) an increased viability, (c) an altered metabolic activity profile, or (d) an altered differentiated functionality particular to the parent cell population.
39. The adapted cell population of embodiment 38 wherein the cell population comprises an immune cell population.
40. The adapted cell population of embodiment 38 wherein the cell population is a candidate or model for a cell therapy product.
41. The adapted cell population of embodiment 38 wherein the cell population is a candidate or model for an autologous cell therapy product.
42. The adapted cell population of embodiment 38 wherein the cell population is a candidate or model for an allogenic cell therapy product.
43. A method of cell banking a cell population, the method comprising:
   freezing a cell population of interest in a liquid cryopreserving medium to form a frozen cell-banked cell population;
   thawing the frozen cell population into a liquid cell culture medium;
   culturing the thawed cell population in a cell culture system that can regulate (a) a total gas atmospheric pressure and (b) a hypoxic gas composition to form a hyperbaric and hypoxic gas environment that surrounds a cell culture container in the system, wherein the total gas atmospheric pressure and the hypoxic gas composition are regulated independently of each other; and
   expanding the cell population within the cell culture system, and within the hyperbaric and hypoxic gas environment, to yield a cell population expanded to a desired level.
44. The method of embodiment 43, wherein prior to freezing the cell population of interest, the method comprises culturing the population in the hyperbaric and hypoxic gas environment provided by the cell culture system.
45. The method of embodiment 43, wherein the cell population of interest is a population derived from a parent population, the cell population of interest being phenotypically distinct from the parent population.
46. The method of embodiment 45, wherein being distinct from the parent population comprises one or more of (a) an increased growth rate, (b) an increased viability, (c) an altered metabolic activity profile, (d) an altered morphology, or (e) an altered functional feature with respect to that of the parent cell population.
47. A pharmaceutical composition comprising a cell population suitable for infusion into a patient, the cell population comprising an adaptation to a hyperbaric and hypoxic condition,
   wherein the adapted cell population is derived from an originating parent cell population not-adapted to a hypoxic and/or a hyperbaric condition,
   wherein the adapted cell population, in transitioning from the originating parent cell population, has been cultured for a sufficient number of generations under a hypoxic and a hyperbaric condition to permit a phenotypic adaptation to TME conditions to occur that underlies adaptation to the hypoxic and a hyperbaric condition,
   wherein the phenotypically adapted derivative cell population comprises phenotypic features that distinguish it from the parent cell population, and
   wherein the phenotypic features of the derivative cell population, as expressed under the hypoxic and hyperbaric condition, that distinguish it from the phenotypic features of the parent cell population (without a history of exposure to the hypoxic and a hyperbaric condition) comprise one or more of (a) an increased growth rate, (b) an increased viability, (c) an altered metabolic activity profile, or (d) an altered differentiated functionality particular to the parent cell population.
48. A modified immune cell population derived from a native immune cell population, modified for increased cytolytic activity towards a cancer cell population relative to the native immune cell population, the modified immune cell population comprising:
   a. decreased expression of a first protein, wherein the first protein comprises HIF2α, granzyme B (GZMB), perforin, FasL, or GNYL; and
   b. an increased expression of a second protein, wherein the protein comprises HIF1α, TRAIL, FasL, GNLY, or GZMM.
49. The modified immune cell population of any of the preceding embodiments, wherein the modified immune cell population does not differ from the native immune cell population with respect to expression of beta-actin.
50. The modified immune cell population of any of the preceding embodiments, further comprising a decreased expression of granzyme B, wherein the decreased expression of granzyme B comprises a 5, 10, 15, 20, 25, 30, 35, 40, 45, 50, 55, 60, 65, 70, 75, 80, 85, 90, or 95% of a native level of granzyme B expression.
51. The modified immune cell population of any of the preceding embodiments, further comprising a decreased expression of perform, wherein the decreased expression of perform comprises a 5, 10, 15, 20, 25, 30, 35, 40, 45, 50, 55, 60, 65, 70, 75, 80, 85, 90, or 95% of a native level of perforin expression.
52. The modified immune cell population of any of the preceding embodiments, further comprising an increased expression of TRAIL, wherein the increased expression of TRAIL comprises a 5, 10, 15, 20, 25, 30, 35, 40, 45, 50, 55, 60, 65, 70, or 75% increase in a native level TRAIL expression.
53. The modified immune cell population of any of the preceding embodiments, further comprising an increased expression of HIF1α, wherein the increased expression of HIF1α comprises a 200, 250, 300, 350, 400, 450, 500, 600, 700, 800, 900, or 1000% increase in a native level HIF1α expression.
54. The modified immune cell population of any of the preceding embodiments, further comprising an decreased expression of HIF2α, wherein the decreased expression of HIF2α comprises a 10, 20, 30, 40, 50, 60, 70, 80, 90, or 100% of a native level of HIF2α expression.
55. The modified immune cell population of any of the preceding embodiments, further comprising a decreased expression of FASL, wherein the decrease in expression of FASL comprises 5, 10, 15, 20, 25, 30, 35, 40, 45, 50, 55, 60, 65, 70, 75, 80, 85, 90, or 95% of a native level of FASL expression.
56. The modified immune cell population of any of the preceding embodiments, further comprising an increased expression of FasL, wherein the increased expression of FasL comprises a 10, 20, 30, 40, 50, 60, 70, 80, 90, or 100, 110, 120, 125, 130, 140, 150, 160, 170, 180, 190, or 200% increase in a native level FasL expression.
57. The modified immune cell population of any of the preceding embodiments, further comprising a decreased expression of GNLY, wherein the decrease in expression of GNLY comprises 5, 10, 15, 20, 25, 30, 35, 40, 45, 50, 55, 60, 65, 70, 75, 80, 85, 90, or 95% of a native level of GNLY expression.
58. The modified immune cell population of any of the preceding embodiments, further comprising an increased expression of GNLY, wherein the increased expression of GNLY comprises 50, 60, 70, 80, 90, or 100, 110, 120, 125, 130, 140, 150, 160, 170, 180, 190, 200, 250, 300, 350, 400, 450, or 500% increase in a native level GNLY expression.
58. The modified immune cell population of any of the preceding embodiments, further comprising an increased expression of GZMM, wherein the increased expression of GZMM comprises 5, 10, 15, 20, 25, 30, 35, 40, 45, 50, 60, 70, 80, 90, or 100, 110, 120, 125, 130, 140, 150, 160, 170, 180, 190, 200, 210, 220, 230, 240, 250, 260, 270, 280, 290, 300, 325, 350, 375, or 400% increase in a native level GZMM expression.
60. The modified immune cell population of any of the preceding embodiments, wherein the modified immune cell population comprises T cells, or wherein the native immune cell population comprises T cells.
61. The modified immune cell population of any of the preceding embodiments, wherein the modified immune cell population comprises natural killer T cells, or wherein the native immune cell population comprises natural killer T cells.
62. The modified immune cell population of any of the preceding embodiments, wherein the modified immune cell population comprises NK-92 cells, or wherein the native immune cell population comprises NK-92 cells.
63. The modified immune cell population of any of the preceding embodiments, further comprising an increased expression of HIF1α, TRAIL, FasL, GNLY, and GZMM.
64. The modified immune cell population of the immediately preceding embodiment further comprising a decreased expression of HIF2α, GZMB, and Perform.
65. The modified immune cell population of the immediately preceding embodiment further comprising an increased expression of HIF1α, TRAIL, FasL, GNLY, and GZMM in the approximate amounts of 610% HIF1α, 125% TRAIL, 150% FasL, 300% GNLY, and 300% GZMM of native expression levels.
66. The modified immune cell population of the immediately preceding embodiment further comprising a decreased expression of HIF2α, GZMB, and Perform in the approximate amounts of 90% HIF2α, 25% GZMB, and 35% Perform of a native level of expression of native expression levels.
67. The modified immune cell population of the immediately preceding embodiment, wherein the modified immune cell population is long term adapted to hypoxic conditions.
68. The modified immune cell population of any of the preceding embodiments, further comprising an increased expression of HIF1α, TRAIL, and GZMM.
69. The modified immune cell population of the immediately preceding embodiment further comprising a decreased expression of FasL, Perform, and GNYL.
70. The modified immune cell population of the immediately preceding embodiment an increased expression of HIF1α, TRAIL, and GZMM in the approximate amounts of 290% HIF1α, 150% TRAIL, and 175% GZMM of native expression levels.
71. The modified immune cell population of the immediately preceding embodiment further comprising a decreased expression of FasL, Perform, and GNYL in the approximate amounts of, 33% FasL, 50% Perform, and 80% GNYL of native expression levels.
72. The modified immune cell population of the immediately preceding embodiment, wherein the modified immune cell population is short term adapted to hypoxic conditions.
73. The modified immune cell population of any of the preceding embodiments, further comprising an increased expression of HIF1α, TRAIL, FasL, GNLY, and GZMM.
74. The modified immune cell population of the immediately preceding embodiment further comprising a decreased expression of HIF2α, GZMB, FasL, and Perforin.
75. The modified immune cell population of the immediately preceding embodiment an increased expression of HIF1α, TRAIL, FasL, GNLY, and GZMM in the approximate amounts of increased expression of 925% HIF1α, 150% TRAIL, 135% FasL, 500% GNLY, and 225% GZMM of native expression levels.
76. The modified immune cell population of the immediately preceding embodiment further comprising a decreased expression of FasL, Perform, GNYL and GZMM in the approximate amounts of 80% HIF2α, 30% Perforin, 500% GNYL and 220% GZMM of native expression levels.
77. The modified immune cell population of the immediately preceding embodiment, wherein the modified immune cell population is long term adapted to hypoxic and hyperbaric conditions.
78. The modified immune cell population of any of the preceding embodiments, further comprising an increased expression of HIF1α, TRAIL, and GZMM.
79. The modified immune cell population of the immediately preceding embodiment further comprising a decreased expression of FasL, GNLY, and Perform.
80. The modified immune cell population of the immediately preceding embodiment an increased expression of HIF1α, TRAIL, and GZMM in the approximate amounts of increased expression of 525% HIF1α, 125% TRAIL, and 175% GZMM of native expression levels.
81. The modified immune cell population of the immediately preceding embodiment further comprising a decreased expression of FasL, GNLY, and Perform in the approximate amounts of 25% FasL, 50% GNLY, and 33% Perform of native expression levels.
82. The modified immune cell population of the immediately preceding embodiment, wherein the modified immune cell population is short term adapted to hypoxic and hyperbaric conditions.
83. The modified immune cell population of any of the preceding embodiments, wherein the modified immune cell population induces cytolysis of cancer cells by death receptor mediated cytotoxicity.
81. The modified immune cell population of any of the preceding embodiments, wherein death receptor mediated cytotoxicity is induced by increased expression of FasL or TRAI1.
82. The modified immune cell population of any of the preceding embodiments, wherein the modified immune cell population exhibits increased cytolytic activity towards cancer cells at ratios 1:1; 2:1; 5:1; and 10:1 of modified immune cells to cancer cells.
83. The modified immune cell population of any of the preceding embodiments, wherein the increased cytolytic activity towards cancer cells comprises inducing cytolysis of 5, 10, 15, 20, 25, 30, 35, 40, 45, 50, 55, 60, 65, 70, 75, 80, 85, 90, 95, 99, or 100% of cancer cells in a solid tumor.

1. A method of producing a CAR-T cell therapy for increased cytotoxicity towards cancer cells, the method comprising:
a. culturing a population of CAR-T cells in a hypoxic environment and a hyperbaric environment;
b. culturing the population of CAR-T cells in the hypoxic environment and the hyperbaric environment for at least 10 generations to produce the CAR-T cell therapy;
c. wherein the CAR-T cell therapy comprises increased cytotoxicity towards cancer cells as compared to the population of CAR-T cells.

2. The method of claim 1, wherein the CAR-T cell therapy is an allogenic CAR-T cell therapy.

3. (canceled)

4. The method of claim 1, wherein the increased cytotoxicity towards cancer cells as compared to the population of CAR-T cells comprises an improvement of at least 15% in cytotoxicity towards cancer cells.

5. The method of claim 1, wherein the increased cytotoxicity towards cancer cells as compared to the population of CAR-T cells comprises an improvement of at least 5% in cytotoxicity towards cancer cells.

6. The method of claim 1, wherein the increased cytotoxicity towards cancer cells as compared to the population of CAR-T cells comprises an improvement of at least 10% in cytotoxicity towards cancer cells.

7. The method of claim 1, wherein the CAR-T cell therapy further comprises: a) an increased growth rate, (b) an increased viability, or c) both, as compared to the population of CAR-T cells.

8-9. (canceled)

10. The method of claim 1, further comprising contacting the population of CAR-T cells with a cancer cell during the culturing of a. or b.

11. The method of claim 1, further comprising contacting the population of CAR-T cells with a population of cancer cells during the culturing of a. or b. at a ratio of at least 1:1 (the population of CAR-T cells: the population of cancer cells, respectively).

12. The method of claim 1, further comprising contacting the population of CAR-T cells with a population of cancer cells during the culturing of a. or b. at a ratio of at least 2:1 (the population of CAR-T cells: the population of cancer cells, respectively).

13. The method of claim 1, further comprising contacting the population of CAR-T cells with a population of cancer cells during the culturing of a. or b. at a ratio of at least 5:1 (the population of CAR-T cells: the population of cancer cells, respectively).

14. The method of claim 1, further comprising contacting the population of CAR-T cells with a population of cancer cells during the culturing of a. or b. at a ratio of at 10:1 (the population of CAR-T cells: the population of cancer cells, respectively).

15-30. (canceled)

31. The method of claim 1, wherein the CAR-T cell therapy further comprises: (a) an increased growth rate, (b) an increased viability, (c) an altered metabolic activity profile, or (d) an altered differentiated functionality, (e) or combinations thereof.

32. The method of claim 1, wherein the CAR-T cell therapy is an autologous CAR-T cell therapy.

33. The method of claim 1, wherein the hypoxic environment comprises up to 3% wt. O₂.

34. The method of claim 1, wherein the hypoxic environment comprises up to 2% wt. O₂.

35-36. (canceled)

37. The method of claim 1, wherein the hyperbaric environment comprises at least 2 psig.

38. (canceled)

39. The method of claim 1, wherein the hyperbaric environment comprises at least 4 psig.

40. The method of claim 1, wherein the CAR-T cell therapy comprises an elevated level of a hypoxia response element activation within a hypoxia-induced factor (HIF) gene.

41. The method of claim 1, wherein the CAR-T cell therapy comprises an elevated expression of a hypoxia-induced factor.

42. (canceled)

43. The method of claim 1, further comprising cryogenically preserving a quantity of the CAR-T cell therapy as a cell bank.

44. The method of claim 43, further comprising thawing the CAR-T cell therapy, culturing the CAR-T cell therapy, administering the CAR-T cell therapy to a subject, or combinations thereof.

45. The method of claim 1, further comprising administering the CAR-T cell therapy to a subject.

46. The method of claim 1, wherein the CAR-T cell therapy comprises an increased expression of HIF1α, TRAIL, FasL, GNLY, GZMM, or combinations thereof.

47. The method of claim 1, wherein the CAR-T cell therapy comprises a decreased expression of HIF2α, GZMB, Perforin, or combinations thereof.

48-92. (canceled)

93. A CAR-T cell therapy adapted to a hypoxic environment and a hyperbaric environment comprising increased cytotoxicity towards cancer cells as compared to a population of the same CAR-T cells which are not adapted to the hypoxic environment and the hyperbaric environment.

94-100. (canceled)

101. The CAR-T cell therapy of claim 93, wherein the CAR-T cell therapy is short term adapted to hypoxic conditions

102. The CAR-T cell therapy of claim 93, wherein the CAR-T cell therapy is long term adapted to hypoxic conditions.

103-112. (canceled)

113. The CAR-T cell therapy of claim 93, wherein the CAR-T cell therapy comprises an increased expression of HIF1α, TRAIL, FasL, GNLY, GZMM, or combinations thereof.

114. The CAR-T cell therapy of claim 93, wherein the CAR-T cell therapy comprises a decreased expression of HIF2α, GZMB, Perforin, or combinations thereof.

115-150. (canceled)
